(19) United States
(12) Patent Application Publication
(10) Pub. No.: US 2008/0311565 A1
(43) Pub. Date: Dec. 18, 2008

(54) Methods and Kits for Detecting Germ Cell Genomic Instability

(75) Inventors: Marijo Kent-First, Starkville, MI (US); Wael Mohamed Abdel Megid, Fitchburg, WI (US); Jeffery Bacher, Madison, WI (US)

Correspondence Address: MICHAEL BEST & FRIEDRICH LLP, ONE SOUTH PINCKNEY STREET, P O BOX 1806, MADISON, WI, 53701, US

(73) Assignee: PROMEGA CORPORATION, Madison, WI (US)

(21) Appl. No.: 11/577,653

(22) PCT Filed: Oct. 24, 2005
(86) PCT No.: PCT/US2005/038179

§ 371 Date: Sep. 12, 2007

Related U.S. Application Data
(60) Provisional application No. 60/621,277, filed on Oct. 22, 2004.
(60) Provisional application No. 60/661,646, filed on Mar. 14, 2005.
(60) Provisional application No. 60/697,778, filed on Jul. 8, 2005.

(51) Int. Cl. C12Q 1/68

(52) U.S. Cl. 435/6

(57) ABSTRACT

Disclosed are methods for detecting microsatellite instability in the germ line of males, methods of assessing risk for developing testicular cancer, methods of evaluating the microsatellite stability of putative cancer or precancerous cells or a tumor, methods for evaluating germ cells for exposure to mutagens, and kits for use in the methods of the invention.

### CROSS-REFERENCE TO RELATED APPLICATIONS

This application claims priority to U.S. provisional applications 60/621,277, filed on Oct. 22, 2004; 60/661,646, filed on Mar. 14, 2005; and 60/697,778, filed on Jul. 8, 2005. This application is being filed simultaneously with an application entitled “Methods and Kits for Detecting Mutations” filed both in the United States and under the Patent Cooperation Treaty and the entirety of the application is incorporated herein by reference.

### STATEMENT REGARDING FEDERALLY SPONSORED RESEARCH

This invention was made with United States government support awarded by ______.

### INTRODUCTION

The germ line is susceptible to damage resulting from pro-mutagenic changes having the potential to generate mutations, including defects in mismatch repair (MMR), recombination errors, and DNA or chromatin fragmentation, specifically DNA strand breaks. Pro-mutagenic changes may be induced, for example, in the abortive apoptosis pathway, by deficiencies in natural processes such as recombination and chromatin packaging that involve the induction of DNA strand breaks, and by oxidative stress. Single and double DNA strand breaks, aneuploidy, mitochondrial mutations, and other indicators of genomic instability (GI) occur with increased frequency in DNA isolated from sperm obtained from sub-fertile men.

Mice having disrupted expression of DNA mismatch repair proteins were found to exhibit somatic tumors and meiotic arrest (Backer, J. S. Curr Genet 28, 499-501 (1995); Baker, S. M. et al. Cell 82, 309-19 (1995)). Nudell et al. reported that, based on sequence analysis, clones of the dinucleotide repeat D1 9S49 from testicular tissue of infertile men with meiotic arrest have increased mutations, relative to control. (Nudell, D. M. & Turek, P. J. Curr Urol Rep 1, 273-81 (2000)). Supporting the connection between genomic instability, mismatch repair defects, and male factor infertility, Martin et al. found a significant increase in the frequency of aneuploidy in the sperm of men that were heterozygous for mutations in the MSH2 mismatch repair gene, compared to controls (Martin et al. Am J Hum Genet 66, 1149-52 (2000)). Maduro et al. reported that DNA amplified by large pool PCR from testis biopsies from azoospermic men diagnosed with Sertoli Cell Only (SCO) exhibited an increased incidence of microsatellite instability in two or more of seven mononucleotide (BAT-26, BAT-40), dinucleotide (D2S123, D17S250, D18S58, D19S49), or trinucleotide (AR, within exon 1 of androgen receptor) repeat loci analyzed (Maduro et al. Mol Hum Reprod 9:61-8 (2003)). In contrast, Maduro et al. reported that men with maturation (meiotic) arrest or hypospermatogenesis did not exhibit significant instability frequency.

There exists a need in the art for improved methods of evaluating germ line specific genomic instability. Detection of genomic instability will allow assessment of risk for testicular cancer, detection of acute exposure to reactive oxygen species (ROS) or mutagens, and monitoring of exposure over time. There is a need in the art to identify microsatellite loci suitable for use in detecting germ line specific genomic instability.

### SUMMARY OF THE INVENTION

In one aspect, the present invention provides methods for detecting genomic instability in a germ cell by obtaining a first DNA sample from a germ cell. The first DNA sample contains at least one microsatellite locus selected from the group consisting of: Y chromosome microsatellite loci; extended mononucleotide repeat loci having at least 41 repeats; and A-rich short tandem repeats having repeating units selected from the group consisting of AAAAG, AAAAC, and AAAAT. The first DNA sample is then contacted with a first primer and a second primer that hybridize to a first DNA sequence and a second DNA sequence, respectively. The first and second DNA sequences flank or partially overlap the at least one microsatellite locus, under conditions that allow amplification of the at least one microsatellite locus to form a first amplification product. The size of the first amplification product is determined and compared to the expected size of the amplification product. A difference between the size of the first amplification product and the expected size of the amplification product is indicative of genomic instability. The expected size of the amplification product can be determined by obtaining a second DNA sample from at least one control cell. This DNA sample is then contacted with the same primers as above and the second DNA sample is amplified and compared to the first DNA sample. The method can be used to detect germ line specific genomic instability and germ line specific genomic instability is indicative of infertility.

In another aspect, the present invention provides methods for detecting genomic instability by obtaining a first DNA sample from a testicular cell. The first DNA sample contains at least one microsatellite locus selected from the group consisting of Y chromosome microsatellite loci, extended mononucleotide repeat loci having at least 41 repeats, MONO-27, NR-24, PENTA D, BAT-25, D7S3070, and D7S1808. The first DNA sample is then amplified as described above to form a first amplification product. The size of the first amplification product is determined and compared to the expected size of the amplification product. A difference between the size of the first amplification product and the expected size of the amplification product is indicative of genomic instability. Genomic instability in testicular cells is indicative of infertility.

In another aspect, the present invention provides methods for assessing risk of testicular cancer. The method involves detecting germ line specific genomic instability by amplifying DNA from germ cells. The DNA contains one or more microsatellite loci that are sensitive to germ line genomic instability. The DNA is amplified as above and the sizes of the amplification products compared to the expected size of the amplification product. Differences between the size of the amplification product and the expected amplification product are indicative of germ line specific genomic instability. Germ line specific genomic instability is indicative of increased risk of testicular cancer.

In another aspect, the present invention provides methods of assessing risk of testicular cancer by obtaining DNA samples from testicular cells. The DNA sample contains one or more microsatellite locus selected from the group consisting of Y chromosome microsatellite loci, extended mononucleotide repeat loci having at least 41 repeats, MONO-27, NR-24, PENTA D, BAT-25, D7S3070, and D7S1808. The DNA sample is then amplified as described above to form a first amplification product. The size of the first amplification product is determined and compared to the expected size of the amplification product. A difference between the size of the first amplification product and the expected size of the amplification product is indicative of genomic instability and genomic instability is indicative of increased risk of testicular cancer.

In yet another aspect, the present invention provides kits for detecting genomic instability and germ line specific genomic instability. The present invention also provides kits for assessing infertility and for assessing the risk of testicular cancer.

In another aspect, the invention provides methods for detecting microsatellite instability in a putative cancer or precancerous cell or a tumor comprising evaluating the stability of Y-chromosome microsatellite loci by methods similar to those previously described. Stability of the putative cancer or precancerous cell or the tumor can be assessed by comparison to a normal cell. Additionally, the present invention also provides kits for detecting microsatellite instability in a putative cancer or precancerous cell or a tumor.

Additionally, the present invention provides methods for monitoring the genomic stability of cultured pluripotent or stem cell lines by obtaining DNA samples from these cells that contain at least one microsatellite locus. The DNA sample is then amplified as described above to form a first amplification product. The size of the first amplification product is determined and compared to the expected size of the amplification product. A difference between the size of the first amplification product and the expected size of the amplification product is indicative of genomic instability. In yet another aspect, the present invention provides kits for determining the genomic stability of cultured pluripotent or stem cell lines.

In yet another aspect, the present invention provides methods for monitoring exposure to mutagens or potential mutagens, including reactive oxygen species, by evaluating the genomic stability of germ cells. A first DNA sample is obtained from at least one germ cell, and the first DNA sample contains at least one microsatellite locus selected from the group consisting of Y chromosome microsatellite loci, extended mononucleotide repeat loci having at least 41 repeats, MONO-27, PENTA C, and D7S3070. The DNA sample is then amplified as described above to form a first amplification product. The size of the first amplification product is determined and compared to the expected size of the amplification product. A difference between the size of the first amplification product and the expected size of the amplification product is indicative of exposure to a mutagen.

In yet another aspect, the present invention provides methods for monitoring exposure to mutagens or potential mutagens, including reactive oxygen species, by evaluating the genomic stability of germ cells. A first DNA sample is obtained from at least one germ cell, and the first DNA sample contains at least one microsatellite locus selected from the group consisting of Y chromosome microsatellite loci, extended mononucleotide repeat loci having at least 38 repeats, MONO-27, PENTA C, and D7S3070. The DNA sample is then amplified as described above to form a first amplification product. A second DNA sample is obtained from at least one control cell either prior to obtaining the first DNA sample or from matched non-exposed cells. The second DNA sample is amplified as described to form a second amplification product. The size of the first and second amplification products are determined and compared. A difference between the size of the first amplification product and the second amplification product is indicative of exposure to a mutagen.

Additionally, the present invention provides kits for monitoring exposure to mutagens or potential mutagens, including reactive oxygen species.

### BRIEF DESCRIPTION OF THE DRAWINGS

FIG. 1 compares the mean mutation frequencies of Y-STR loci and mononucleotide repeats with extended polyA tracts in irradiated cells.

FIG. 2 shows the frequency of genomic instability for each tested loci in sperm from a group of infertile men and a subpopulation within that group having a relatively high microsatellite instability.

FIG. 3 compares the percent of genomic instability in infertile men for two different panels of loci.

FIG. 4 shows the distribution of percent genomic instability (white bars) and the sperm cells concentrations in millions/ml (black bars) among tested infertile men.

FIG. 5 is a bar graph depicting the distribution of individuals classified as MSI-High, MSI-Intermediate, MSI-Low, or MSI-Stable among infertile men in groups 1-5, and among men in a fertile control group.

FIG. 6 shows the percent DNA fragmentation index and sperm cell concentrations in millions/ml of samples from infertile men in groups 2-5.

### DETAILED DESCRIPTION

Nearly one third of the human genome is composed of DNA repeats. The Y-chromosome contains the largest clusters of repetitive elements, including tandem and interspersed repeats and palindromes of elements that include short tandem repeats (STRs), genes and sequence tagged sites (STS). With the exception of the Pseudoautosomal Pairing Regions (PAR) adjacent to the telomeres, the Y chromosome does not undergo recombination. Therefore, mutations in the Non-Recombining regions of the Y-chromosome (NRY) are not subject to many of the DNA repair mechanisms that other chromosomes with pairing homologues utilize to repair mutations in noncoding regions. In males, the X chromosome has no pairing homologue and therefore it also does not undergo recombination and does not have the benefit of the DNA repair mechanisms that other chromosomes utilize to repair mutations in noncoding regions.

Many of the genes required for spermatogenesis are encoded on the Y chromosome. Prior studies have demonstrated that radiation exposure of 1.5 Gy or more often results in persistent azoospermia or reduced sperm production, presumably due to deletions encompassing genes necessary for spermatogenesis (Birioukov, et al. Arch Androl 1993 30(2):99-104; Greiner Strahlenschutz Forsch Prax 1985 26:114-121, which are incorporated herein by reference). Germline mutation rates in short tandem repeats on the Y chromosome are similar to those observed on autosomal chromosomes (i.e., about 1.6×10⁻³) (Bodowle, et al. Forensic Science International 2005 150(l):1-15, which is incorporated herein by reference in its entirety).

The present invention provides methods for detecting genomic instability. Genomic instability is indicated by length variations in microsatellite loci which indicate mutations occurred in the loci. Microsatellite loci comprise extended mononucleotide repeat loci and short tandem repeats, particularly short tandem repeats on the Y chromosome. The present invention provides methods for assessing germ line specific genomic instability and infertility by observing allelic length variations in mononucleotide repeat tracts or in certain short tandem repeats comprising repeating units of 1-6 base pairs in germ cells or testicular cells as compared to control cells of the same individual. Assessment of germ line specific genomic instability can also be used to assess the risk of testicular cancer. The present invention also provides methods for evaluating microsatellite instability in putative cancer or precancerous cells, tumor cells, pluripotent cells or cultured stem cells. Finally, the present invention provides a method of monitoring exposure to mutagens, such as ROS, by evaluating microsatellite stability in germ cells.

Repetitive DNA sequences (or “DNA repeats”) have been identified that are susceptible to mutation in response to mutagens. Microsatellite loci are a class of DNA repeats, each of which contains a sequence of 1-9 base pairs (bp) that is tandemly repeated. Loci having larger repeat units of 10 to 60 bp are typically referred to as minisatellites. Microsatellites and minisatellites are inherently unstable and mutate at rates several orders of magnitude higher than non-repetitive DNA sequences. Due to this instability, microsatellites and minisatellites were evaluated for increased mutation rates after exposure to mutagens, inducers of free radicals, and ROS.

As used herein, “mutagen” refers to a substance or condition that causes a change in DNA including, but not limited to, chemical or biological substances, for example, free radicals, reactive oxygen species (ROS), drugs, chemicals, radiation and the normal aging process. By “exposing” it is meant contacting a cell or organism with a mutagen or treating a cell or organism under conditions that result in interaction of the cell or organism with a mutagen. It should be understood that “exposing” a cell or organism to a mutagen does not necessarily require an active step. Rather, exposure of a cell or organism to a mutagen may result from the cell or organism being present in an environment in which the mutagen occurs.

Briefly, the method involves amplifying a DNA sample comprising one or more microsatellite locus using primers that hybridize to DNA sequences that flank or partially overlap the microsatellite locus in an amplification reaction, suitably a polymerase chain reaction (PCR). The upper limit of the size of the DNA sequence to be amplified will depend on the efficiency of the amplification method. The size of the DNA sequence may be selected to reduce length variations due to incomplete copying of the target DNA sample and a high fidelity polymerase may be used to decrease the chance of PCR artifacts. Suitably, the DNA sequence to be amplified is at most about 1000 base pairs in length.

As described in the Examples below, a number of microsatellite loci were identified as being sensitive to ionizing radiation or oxidative stress caused by increases in ROS. Those same loci exhibit increased germ line specific genomic instability in individuals with spermatogenic failure, relative to individuals with normal spermatogenesis. In particular, microsatellite loci on the Y chromosome (or Y chromosome short tandem repeat loci (YSTRs)), extended mononucleotide repeat loci (monoucleotide repeats containing at least 38 nucleotides), and A-rich pentanucleotide repeat loci are sensitive to ROS and to ionizing radiation, and are predictive of germ line specific genomic instability. For example, the A-rich autosomal pentanucleotide repeat loci Penta C and Penta D, which contain the motif AAAAG repeated 15 and 17 times, respectively, were found to be sensitive to ROS. Penta D exhibited greater instability in germ cells of infertile men than did the Penta C. The differential sensitivity may be a function of the number of repeats. Sensitivity of pentanucleotide repeats to ROS and germ line specific mutation is surprising in that pentanucleotide repeats are relatively stable in MMR deficient tumors and in fact, are used as a control in detecting MSI in MMR deficient cells.

In addition to those YSTR loci exemplified below as exhibiting sensitivity to ROS or germ line specific genomic instability (i.e., DYS438, DYS389-II, DYS390, DYS439, DYS392, DYS385b, DYS19, DYS389-I, DYS385a, DYS393, and DYS437), it is reasonably expected that other YSTR loci of the NRY will be suitable for detecting ROS exposure or germ line specific genomic instability, including, but are not limited to, DYS453, DYS456, DYS446, DYS455, DYS463, DYS435, DYS458, DYS449, DYS454, DYS434, DYS437, DYS435, DYS439, DYS488, DYS447, DYS436, DYS390, DYS460, DYS461, DYS462, DYS448, DYS452, DYS464a, DYS464b, DYS464c, DYS464d, DYS459a, and DYS459b (see Table 9). These Y chromosome microsatellite loci were identified in a search of available sequence information, but any other mono-, di-, tri-, tetra-, or pentanucleotide repeat on the NRY of the Y chromosome is expected to be suitable in the methods of the current invention.

In the examples below, several extended mononucleotide repeat loci were also demonstrated to exhibit germ line specific genomic instability and/or sensitivity to mutagens such as ROS (i.e. hBAT-51d, hBAT-52a, hBAT-53c, hBAT59a, hBAT-60a, hBAT-60b and hBAT-62). It is reasonably expected that other extended mononucleotide repeat loci will be suitable for detecting ROS exposure or germ line specific genomic instability, including, but not limited to, those loci listed in Table 3. These extended mononucleotide repeat loci were identified in a search of available sequence information, but any other extended mononucleotide repeat loci having at least 38 repeats is suitable for use in the methods of the present invention. Suitably, the extended mononucleotide repeat loci will contain between 38 and 200 repeats, between 41 and 200 repeats, between 38 and 90 repeats, between 41 and 90 repeats, between 42 and 90 repeats or between 42 and 60 repeats.

Mutational load profiling, through analysis of changes in microsatellite repeat sequences over time, is a non-invasive and generalized approach for monitoring an individual's cumulative record of mutations. This approach is useful in predicting and minimizing health risks for individuals exposed to mutagens. The methods of the invention can be used measure genetic damage from drugs on experimental cell cultures or whole animals.

As demonstrated below, a number of loci comprising repetitive DNA sequences were found to be unstable in the germ line of infertile men, but are stable in control somatic cells and in the germ line of fertile men. Therefore, these loci are useful in evaluating germ line specific genomic instability. Detection of germ line specific genomic instability in these loci may be used in diagnosing, treating, or assessing the prognosis of individuals seeking help for infertility or risk of testicular cancer. For example, the methods may be used to evaluate chances of successful in vitro fertilization or in preimplantation diagnostic testing. Several microsatellite loci were shown to be suitable for evaluating genomic instability. These loci include DYS438, DYS389-II, DYS390, DYS439, DYS392, DYS385b, DYS19, DYS389-I, DYS385a, DYS393, DYS437, BAT-40, MONO-27, NR-24, PENTA D, BAT-25, BAT-26, D7S3070, and D7S1808. It is expected that other microsatellite loci will be suitable in the methods of the invention.

As used herein, loci that are unstable in the germ line of infertile men are those loci that are unstable in at least 5% of infertile men with spermatogenic arrest and in less than 5%, suitably less than 2%, 1% or 0% of fertile men. Preferably, the unstable locus is unstable in at least 10%, 15%, 20%, 25%, or 30% or more of infertile men with spermatogenic arrest. In the Examples, genomic instability was measured by evaluating the sizes of amplification products and deducing the presence of mutant alleles by comparing the size of the amplified product from a germ cell or testicular cell or tissue to that of somatic control cells (e.g., lymphocytes) or the expected size of the amplified product.

Analysis of an amplification product involves comparing the size of the amplification product to the expected size of the amplification product. The expected size of the amplified product can be established by comparison to the amplification product derived from control cells. The control cells can be somatic cells from the same individual as germ cells or cells of the same individual taken at a different (e.g., earlier) time point. Control cells can also be matched cells from an inbred population of organisms, a tissue culture cell line or an unexposed portion of an organism. If a microsatellite locus has a predominant allele in the population, then the expected size of the amplification product can be established by comparison to the size of the locus in the population. Finally, the expected size of the amplification product can be established by pedigree analysis.

In the Examples, the sizes of amplified products were evaluated by capillary electrophoresis. However, the sizes of the amplified products may be assessed by any suitable means, e.g., sequencing alleles, or by observing increased or decreased expression of reporter proteins in cells containing a DNA construct comprising a reporter gene fused to a DNA repeat such that alterations in the length of the DNA repeat result in a frame shift and loss or gain of reporter gene expression, as described in U.S. Patent Application No. ______ entitled “Methods and Kits for Detecting Mutations,” filed Oct. 24, 2005, which is incorporated herein by reference.

When evaluating genomic instability by amplifying the loci, the loci may be amplified and analyzed individually, or in combination with other loci as part of a panel. Inclusion of multiple loci in a panel increases the sensitivity of the panel. Suitably, at least four different loci are evaluated for genomic instability. Preferably, at least five loci are evaluated for genomic instability. Multiple loci may be amplified separately or, conveniently, may be amplified together with other loci in a multiplex reaction.

Suitably, one or more Y-linked monomeric, dimeric, trimeric, tetrameric, or pentameric repeats are included in the panel for evaluating germ line specific genomic instability. The Y-linked repeat may suitably be associated with the non-recombining regions of the Y chromosome. Autosomal pentanucleotide repeat loci are also suitable for detecting germ line specific genomic instability. Extended mononucleotide repeat loci, preferably containing adenine repeats, are also suitable for detecting germ line specific genomic instability. Extended mononucleotide repeat loci, as used herein, refer to mononucleotide repeats of at least 38 nucleotides per repeat unit. Extended mononucleotide repeat loci suitably have repeats of between 38 and 200 nucleotides, between 41 and 200 nucleotides, between 38 and 90 nucleotides, between 41 and 90 nucleotides, between 42 and 90 nucleotides or between 42 and 60 nucleotides.

In amplifying a repeat locus according to the methods of the invention, one may use any suitable primer pair, including, for example, those described herein below or those available commercially (e.g., PowerPlex®Y System, Promega Corporation, Madison, Wis.). Alternatively, one may design suitable primer pairs that are adjacent to or which partially overlap each end of the locus to be amplified using available sequence information and software for designing oligonucleotide primers, such as Oligo Primer Analysis Software version 6.86 (National Biosciences, Plymouth, Minn.).

Germ cells may be obtained by any suitable means, including collecting sperm cells from ejaculated semen or from aspirates of semeniferous tubules and/or the epididymis, testicular biopsy, egg harvest, pluripotent or stem cells isolated from biological samples, cultured pluripotent cells, or cultured stem cells. Similarly, DNA to be amplified may be isolated by any suitable means. The DNA to be amplified may be from a single cell, small pool DNA, or large pool DNA. DNA from a single cell may be amplified by whole genome amplification.

Following evaluation of microsatellite instability, individuals tested were assigned to MSI classifications based on the percentage of tested loci that exhibit instability. Those having high MSI (±30% of loci) were designated MSI-H; those having intermediate MSI (20-29% of loci) were designated MSI-I; those having low MSI (5-19%) were designated MSI-L; and those having no MSI were designated MSS for microsatellite stable. As detailed in the Examples, a relatively large percentage of infertile men with high or intermediate MSI in their germ cells subsequently developed testicular cancer (seminoma). Therefore, there appears to be a subset of men with germ line specific genomic instability at risk for developing testicular cancer. Using the methods of the invention, it will be possible to identify MSI-H or MSI-I individuals who may require monitoring for testicular cancer.

Historically, testicular cancer is diagnosed only after a testicular mass is appreciated and then biopsied. The discovery that high or intermediate levels of microsatellite instability of certain loci in germ cells is correlated with increased risk of testicular cancer will permit early detection (i.e., prior to the development of an appreciable mass) of this type of cancer. The methods of the invention can be performed on samples obtained by non-invasive means (e.g., ejaculated sperm cells or sperm cells obtained by fine needle aspiration), relative to conventional tissue biopsy. These factors are likely to promote early detection and treatment, which greatly improves prognosis.

In the Examples, several different colon cancer samples were evaluated for MSI using the Y chromosome microsatellite markers. Previous studies had demonstrated that in individuals with hereditary non-polyposis colorectal cancer (HNPCC), who carry germline mutations in DNA mismatch repair genes including MLH1 and MSH2, mononucleotide repeats are mutated more frequently in mismatch repair (MMR) deficient cancer cells. Detection of increased microsatellite instability in these tumor cells provides important diagnostic information relevant to treatment and prognosis. As illustrated in the Examples, several Y-chromosome microsatellite loci were shown to be mutated in mismatch repair deficient tumors, but not in mismatch repair proficient tumors. The loci tested included DYS438, DYS389-II, DYS390, DYS439, DYS392, DYS385b, DYS19, DYS389-I, DYS385a, DYS393, and DYS437. The ability to distinguish between mismatch repair deficient and proficient tumors is important in diagnosis and treatment of cancers. Because each of the Y-STR loci is associated with non-recombining regions of the Y-chromosome, it is envisioned that other microsatellite loci of the NRY may be suitable for use in distinguishing between mismatch repair deficient and proficient tumors in males, including, but not limited to, DYS453, DYS456, DYS446, DYS455, DYS463, DYS435, DYS458, DYS449, DYS454, DYS434, DYS437, DYS435, DYS439, DYS488, DYS447, DYS436, DYS390, DYS460, DYS461, DYS462, DYS448, DYS452, DYS464a, DYS464b, DYS464c, DYS464d, DYS459a, and DYS459b.

The methods of the present invention may also be used to detect microsatellite instability in putative cancer or precancerous cells or in a tumor. The Y chromosome microsatellite loci may be suitable for use in distinguishing microsatellite stable and unstable cells. This distinction is significant to the diagnosis and prognosis of putative cancer or precancerous cells and tumors. Cells may be considered putative cancer or precancerous if the cells appear atypical microscopically, in culture or are contained in a polyp or other abnormal mass. Microsatellite stability can be assessed by comparison of the amplification products from these cells to matched amplification products from normal cells. Normal cells are cells that are microsatellite stable and do not exhibit any precancerous characteristics, such as normal blood lymphocytes.

The present invention provides kits for performing the methods of the invention. These kits may contain one or more primers or primer pairs, buffers for isolating DNA or for performing amplification reactions, and/or instructions for carrying out the methods of the invention.

The following non-limiting Examples are intended to be purely illustrative.

### EXAMPLES

### A. Detection of Mutations in Radiation Treated Cultured Human Fibroblast and Cell Lines.

Cell culture and irradiation. Male human fibroblast cell line No. AG01522 from Coriell Cell Repository was grown in MEM Eagle-Earle BSS media with 15% fetal bovine serum and 2× concentration of essential and non-essential amino acids and vitamins with 2 mM L-glutamine. Cell cultures were grown at 37° C. and 5% CO₂ under sterile conditions. Exponentially growing cells were plated in T-25 tissue culture flasks and were irradiated at room temperature with a single dose 0.5, 1 or 3 Gy of 1 GeV/nucleon ⁵⁶Fe ions accelerated with the Alternating Gradient Synchrotron (AGS) at the Brookhaven National Laboratory at a rate of 0.5 Gy/min. Following irradiation, media was replaced and cells grown for 3 days then collected and frozen at −70° C. until ready for DNA extraction.

Small-pool PCR amplification of microsatellite repeats. Small-pool PCR (SP-PCR) amplification of loci including mononucleotide repeat markers NR-21, NR-24, BAT-25, BAT-26 and MONO-27, tetranucleotide repeat markers on autosomal chromosomes (D7S3070, D7S3046, D7S1808, D10S1426 and D3S2432), tri-, tetra- and penta-nucleotide repeats on the Y chromosome (DYS391, DYS389 I, DYS389 II, DYS438, DYS437, DYS19, DYS392, DYS393, DYS390, and DYS385), penta-nucleotide repeats Penta B, C, D, and E, and mononucleotide repeat loci with extended polyA tracts (hBAT-5 1d, hBAT-52a, hBAT-53c, hBAT59a, hBAT-60a, hBAT-60b and hBAT-62) was performed using fluorescently labeled primer pairs for each loci (Table 1). PCR reactions were performed by using 6-15 pg of total genomic DNA in a 10 μl reaction mixture containing 1 μl Gold ST*R 10× Buffer (Promega, Madison, Wis.), 0.05 μl AmpliTaq gold DNA polymerase (5 units/μl; Perkin Elmer, Wellesley, Mass.) and 0.1-10 μM each primer. PCR was performed on a PE 9600 Thermal Cycler (Applied Biosystems, Foster City, Calif.) using the following cycling conditions: initial denaturation for 11 min at 95° C. followed by 1 cycle of 1 min at 96° C., 10 cycles of 30 sec at 94° C., ramp 68 sec to 58° C., hold for 30 sec, ramp 50 sec to 70° C., hold for 60 sec, 25 cycles of 30 sec at 90° C., ramp 60 sec to 62° C., hold for 30 sec, ramp 50 sec to 70° C., hold for 60 sec, final extension of 30 min at 60° C. and hold at 4° C. The SP-PCR products were separated and detected by capillary electrophoresis using an Applied Biosystems 3100 Genetic Analyzer and data analyzed using AB GeneScan and Genotyper Software Analysis packages to identify presence of microsatellite mutations.

TABLE 1

| | | | | 5′ | |
|---|---|---|---|---|---|
| Locus | Repeats | Chromosome | Oligonucleotide Sequence | end | |
| DYS393 | (AGAT) | Y | GTG GTC TTC TAC TTG TGT CAA TAC AG | TMR | SEQ ID NO:1 |
| | | | GAA CTC AAG TCC AAA AAA TGA GG | OH | SEQ ID NO:2 |
| DYS390 | (TCTG)/(TCTA) | Y | ATT TAT ATT TTA CAC ATT TTT GGG CC | OH | SEQ ID NO:3 |
| | | | TGA CAG TAA AAT GAA AAC ATT GC | TMR | SEQ ID NO:4 |
| DYS385 | GMA | Y | ATT AGC ATG GGT GAC AGA GCT A | OH | SEQ ID NO:5 |
| | | | CCA ATT ACA TAG TCC TCC TTT C | TMR | SEQ ID NO:6 |
| DYS391 | (TCTA) | Y | TTC AAT CAT ACA CCC ATA TCT GTC | FL | SEQ ID NO:7 |
| | | | ATT ATA GAG GGA TAG GTA GGC AG | OH | SEQ ID NO:8 |
| DYS389I/II | (TCTG)/(TCTA) | Y | CCA ACT CTC ATC TGT ATT ATC TAT G | FL | SEQ ID NO:9 |
| | | | ATT TTA TCC CTG AGT AGC AGA AGA ATG | OH | SEQ ID NO:10 |
| DYS439 | GATA | Y | TCG AGT TGT TAT GGT TTT AGG | FL | SEQ ID NO:11 |
| | | | ATT TGG CTT GGA ATT CTT TTA CCC | OH | SEQ ID NO:12 |
| DYS438 | (TTTTC) | Y | TGG GGA ATA GTT GAA CGG TA | JOE | SEQ ID NO:13 |
| | | | ATT GCA ACA AGA GTG AAA CTC CAT T | OH | SEQ ID NO:14 |
| DYS437 | (TCTA/(TCTG) | Y | ATT GAC TAT GGG CGT GAG TGC AT | OH | SEQ ID NO:15 |
| | | | AGA CCC TGT CAT TCA CAG ATG A | JOE | SEQ ID NO:16 |
| DYS19 | (TAGA) | Y | ACT ACT GAG TTT CTG TTA TAG TGT TTT T | JOE | SEQ ID NO:17 |
| | | | GTC AAT CTC TGC ACC TGG AAA T | OH | SEQ ID NO:18 |
| DYS392 | (TAT) | Y | ATT TAG AGG CAG TCA TCG CAG TG | OH | SEQ ID NO:19 |
| | | | ACC TAC CAA TCC CAT TCC TTA G | JOE | SEQ ID NO:20 |
| NR-21 | (A) | 14 | CGGAGTCGCTGGCACAGTTCTATT | JOE | SEQ ID NO:21 |
| | | | TCGCGTTTACAAACAAGAAAAGTGT | OH | SEQ ID NO:22 |
| BAT-26 | (A) | 2 | TGACTACTTTTGACTTCAGCCAGT | FL | SEQ ID NO:23 |
| | | | AACCATTCAACATTTTTAACCCTT | OH | SEQ ID NO:24 |
| BAT-25 | (A) | 4 | TCGCCTCCAAGAATGTAAGT | JOE | SEQ ID NO:25 |
| | | | ATTTCTGCATTTTAACTATGGCTC | OH | SEQ ID NO:26 |
| NR-24 | A | 2 | CCATTGCTGAATTTTACCTC | TMR | SEQ ID NO:27 |
| | | | ATTGTGCCATTGCATTCCAA | OH | SEQ ID NO:28 |
| MONO-27 | (A0 | 2 | TGTGAACCACCTATGAATTGCAGA | JOE | SEQ ID NO:29 |
| | | | ATTGCTTGCAGTGAGCAGAGATCGTT | OH | SEQ ID NO:30 |
| Penta C | (AAAAG) | 9 | CATGGCATTGGGGACATGAACACA | TMR | SEQ ID NO:31 |
| | | | CACTGAGCGCTTCTAGGGACTTCT | OH | SEQ ID NO:32 |
| Penta D | (AAAAG) | 21 | CAGCCTAGGTGACAGAGCAAGACA | FL | SEQ ID NO:33 |
| | | | ATTTGCCTAACCTATGGTCATAAC | OH | SEQ ID NO:34 |
| hBAT-51d | (A) | Y | GAGGCTGAGGCAGGAGAATGGCGTGAAC | FL | SEQ ID NO:35 |
| | | | CGCTGACGCAGAACCTGAAATTGTGATT | OH | SEQ ID NO:36 |
| hBAT-53C | A | Y | TATCCTAGCTTGGCCTGTTTAAGACC | JOE | SEQ ID NO:37 |
| | | | TGAGGCAGGAGAATGGCGTGAA | OH | SEQ ID NO:38 |
| hBAT-60A | (A) | 8 | TCTCATTTGAGTGGTGGAAGTGACTGGT | JOE | SEQ ID NO:39 |
| | | | TATTCTTTCGGGATGTAATCTCT | OH | SEQ ID NO:40 |
| hBAT-62 | (A) | 2 | AGGCTGAAGCAGGAGAATCACTTAAAAC | JOE | SEQ ID NO:41 |
| | | | GCCAAGTGTCGCTTGTAATTCTATT | OH | SEQ ID NO:42 |
| hBAT-52A | (A) | X | CTAACTTCCCAGCAACTTCCTTTACACT | FL | SEQ ID NO:43 |
| | | | ATTGGGCAGACACTGAACTAGCTT | OH | SEQ ID NO:44 |
| hBAT-59A | (A) | 12 | CAGCCTAGGTAACAGAGCAAGACCTTTG | FL | SEQ ID NO:45 |
| | | | GTTTGCGTGATTTGCGTGGACTT | OH | SEQ ID NO:46 |
| hBAT-56a | (A) | X | TCAGCAGCTGAAAGAAATCTGAGTAC | JOE | SEQ ID NO:47 |
| | | | GCGATACCCAAAGTCAATAGTC | OH | SEQ ID NO:48 |
| hBAT-56b | (A) | X | GAAGCTGCAGTAAGCCGAGATTGT | FL | SEQ ID NO:49 |
| | | | GCCCTCTTAACTCCCATGACATTC | OH | SEQ ID NO:50 |
| D7S3070 | (GATA) | | CATTTCTTCTGCCCCCATGA | | SEQ ID NO:51 |
| | | | attTGACAGCTGAAAAGGTGCAGATG | | SEQ ID NO:52 |
| D7S3046 | (GATA) | | GAGGAGACAGCCAGGGATATA | | SEQ ID NO:53 |
| | | | attTCTCTATAACCTCTCTCCCTATCT | | SEQ ID NO:54 |
| D7S1808 | (GGAA) | | GGAGGAAAAGTCTTAAACGTGAAT | | SEQ ID NO:55 |
| | | | attGGCCTTGATGTGTTTGTTACT | | SEQ ID NO:56 |
| D10S1426 | (GATA) | | GCCGATCCTGAAGCAATAGC | | SEQ ID NO:57 |
| | | | attCCCCTTGGTGGTGTCATCCT | | SEQ ID NO:58 |
| D3S2432 | (GATA) | | GTTTGCATGTGAACAGGTCA | | SEQ ID NO:59 |
| | | | attGGCAGGCAGGTAGATAGACA | | SEQ ID NO:60 |
| FGA | (TTTC) | 4 | GGCTGCAGGGCATAACATTA | TMR | SEQ ID NO:61 |
| | | | ATTCTATGACTTTGCGCTTCAGGA | OH | SEQ ID NO:62 |
| TPOX | (AATG) | 2 | GCACAGAACAGGCACTTAGG | OH | SEQ ID NO:63 |
| | | | CGCTCAAACGTGAGGTTG | TMR | SEQ ID NO:64 |
| D8S1179 | (TCTA) | 8 | ATTGCAACTTATATGTATTTTTGTATTTCATG | OH | SEQ ID NO:65 |
| | | | ACCAAATTGTGTTCATGAGTATAGTTTC | TMR | SEQ ID NO:66 |
| vWA | (TCTA) | 12 | GCCCTAGTGGATGATAAGAATAATCAGTATGTG | OH | SEQ ID NO:67 |
| | | | GGACAGATGATAAATACATAGGATGGATGG | TMR | SEQ ID NO:68 |
| Amelogenin | | X | CCCTGGGCTCTGTAAAGAA | TMR | SEQ ID NO:69 |
| | | | ATCAGAGCTTAAACTGGGAAGCTG | OH | SEQ ID NO:70 |
| Penta E | (AAAGA) | 15 | ATTACCAACATGAAAGGGTACCAATA | OH | SEQ ID NO:71 |
| | | | TGGGTTATTAATTGAGAAAACTCCTTACAATTT | FL | SEQ ID NO:72 |
| D18S51 | (AGAA) | 18 | TTCTTGAGCCCAGAAGGTTA | FL | SEQ ID NO:73 |
| | | | ATTCTACCAGCAACAACACAAATAAAC | OH | SEQ ID NO:74 |
| D21S11 | (TCTA) | 21 | ATATGTGAGTCAATTCCCCAAG | OH | SEQ ID NO:75 |
| | | | TGTATTAGTCAATGTTCTCCAGAGAC | FL | SEQ ID NO:76 |
| TH01 | (AATG) | 11 | GTGATTCCCATTGGCCTGTTC | FL | SEQ ID NO:77 |
| | | | ATTCCTGTGGGCTGAAAAGCTC | OH | SEQ ID NO:78 |
| D3S1358 | (TCTA) | 3 | ACTGCAGTCCAATCTGGGT | OH | SEQ ID NO:79 |
| | | | ATGAAATCAACAGAGGCTTGC | FL | SEQ ID NO:80 |
| Penta D | (AAAGA) | 21 | GAAGGTCGAAGCTGAAGTG | JOE | SEQ ID NO:81 |
| | | | ATTAGAATTCTTTAATCTGGACACAAG | OH | SEQ ID NO:82 |
| CSF1PO | (AGAT) | 5 | CCGGAGGTAAAGGTGTCTTAAAGT | JOE | SEQ ID NO:83 |
| | | | ATTTCCTGTGTCAGACCCTGTT | OH | SEQ ID NO:84 |
| D16S539 | (GATA) | 16 | GGGGGTCTAAGAGCTTGTAAAAAG | OH | SEQ ID NO:85 |
| | | | GTTTGTGTGTGCATCTGTAAGCATGTATC | JOE | SEQ ID NO:86 |
| D7S820 | (GATA) | 7 | ATGTTGGTCAGGCTGACTATG | JOE | SEQ ID NO:87 |
| | | | GATTCCACATTTATCCTCATTGAC | OH | SEQ ID NO:88 |
| D13S317 | (TATC) | 13 | ATTACAGAAGTCTGGGATGTGGAGGA | OH | SEQ ID NO:89 |
| | | | GGCAGCCCAAAAAGACAGA | JOE | SEQ ID NO:90 |
| D5S818 | (AGAT) | 5 | GGTGATTTTCCTCTTTGGTATCC | OH | SEQ ID NO:91 |
| | | | AGGCACAGTTTACAACATTTGTATCT | JOE | SEQ ID NO:92 |

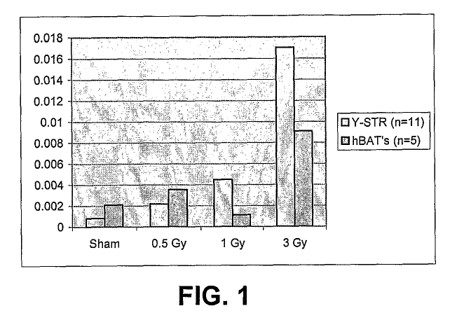

Mutational analysis. Mutations detected in microsatellite repeats of DNA isolated from cells irradiated with 0.5, 1 or 3 Gy iron ions are summarized in Table 2. Mononucleotide repeats with polyA runs of less than 36 bp exhibited little or no increase in mutation rates over controls. Similarly, tetranucleotide repeats on autosomal chromosomes that are sensitive to MSI did not exhibit any evidence of radiation-induced mutations. In contrast, A-rich pentanucleotide repeats and repeats on the Y chromosome did show statistically significant increases in mutations in irradiated cells. FIG. 1 shows the mean mutation frequencies of loci in the Y-STR panel and mononucleotide repeats with extended polyA tracts in irradiated human cells on exposure to various doses of radiation. One-way ANOVA showed significant increases in mutation frequencies in Y-STRs following exposure of human fibroblasts to 3 Gy and 1 Gy and in hBATs following exposure to 3 Gy as compared to the sham (p<0.001).

TABLE 2
Mutational analysis of human cultured fibroblast cells following exposure to ionizing radiation.

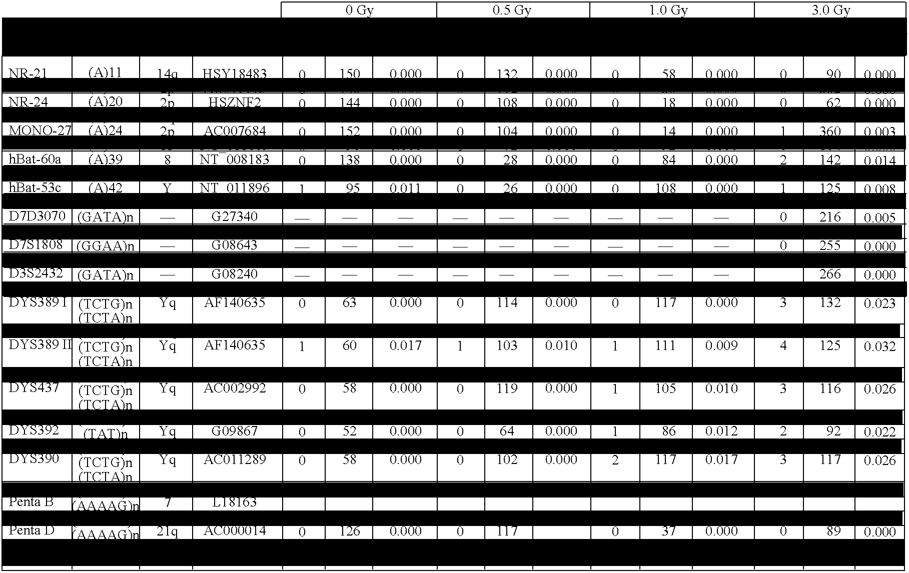

Dose-response curves. A linear dose response was observed for microsatellite markers tested on the Y chromosome. Normal human fibroblast cells AG01522 were irradiated with 0, 0.5, 1 or 3 Gy iron ions and the combined mutation frequency of 13 microsatellite markers on the Y chromosome was determined by SP-PCR and plotted as a function of dose. There was a good fit to a linear regression line (R²=0.9835), indicating that these markers would be useful for biodosimetry.

Further details regarding the effect of irradiation on the genomic stability of cultured cells can be found in U.S. Provisional 60/661,646, filed Mar. 14, 2005, which is incorporated by reference in its entirety.

### B. Detection of Mutations in Human Cultured Cells Exposed to Oxidative Stress

Cell culture. Male human fibroblast cell line #AG01522 from Coriell Cell Repository was cultured in MEM Eagle-Earle BSS 2× concentration of essential and non-essential amino acids and vitamins with 2 mM L-glutamine and 15% fetal bovine serum. Cell cultures were grown at 37° C. and 5% CO₂ under sterile conditions and split at a ratio of 1:5 when cells were confluent by releasing cells with trypsin-EDTA treatment. Cells were treated with hydrogen peroxide at concentrations of 0.0 mM, 0.04 mM, 0.4 mM, 0.8 mM, 1.2 mM, and 4 mM in PBS for 1 hour at the same culture conditions described. After treatment, media with hydrogen peroxide was replaced with fresh media and allowed to recover for 3 days. Cells were pelleted and DNA extracted.

Mutation Detection. Mutant alleles were identified by small-pool PCR as described above using microsatellite markers including: mononucleotide repeat markers (NR-21, NR-24, BAT-25, BAT-26 and MONO-27), tetranucleotide repeat markers on autosomal chromosomes (D7S3070, D7S3046, D7S1808, D10S1426 and D3S2432), tri-, tetra- and penta-nucleotide repeats on the Y chromosome (DYS391, DYS389 I, DYS389 II, DYS438, DYS437, DYS19, DYS392, DYS393, DYS390, and DYS385), penta-nucleotide repeats Penta B, C, D, and E, and mononucleotide repeats having extended polyA tracts (hBAT-51d, hBAT-53C, hBAT-60A, hBAT-62, hBAT-52A, and hBAT-59A). Mutations were detected in the mononucleotide repeats having extended polyA tracts, Y-STRs and A-rich pentanucleotide repeats in DNA isolated from cells exposed hydrogen peroxide. Mutation rates of mononucleotide repeats having extended polyA tracts, Y-STRs and A-rich pentanucleotide repeats following exposure to ROS are also dose dependent.

Further details regarding the effect of oxidative stress on the genomic stability of cultured cells can be found in U.S. Provisional 60/661,646, filed Mar. 14, 2005, which is incorporated by reference in its entirety.

### C. Detection of Genomic Instability in Human Germ Line.

Sample acquisition. Samples from clinically selected men or fertile men were collected using standard methods. Assignment to the fertile group was made according to WHO standards or Krueger's strict criteria. Clinically selected participants were profiled using a standardized questionaire administered by the referring treatment centers. Testis phenotype was determined using standard measurable parameters used to clinically diagnose testis function, namely, sperm counts, morphology, motility, testis volume, and reproductive hormones (FSH, LH, and testosterone). In addition, testis histopathology was determined for those individuals with azoospermia or severe oligozoospermia. Based on these criteria, infertile individuals were assigned to one of five infertile groups, which include individuals with non-obstructive azoospermia (Groups 1a and 1b), severe oligozoospermia (Group 2), moderate oligozoospermia (Group 3), mild oligozoospermia (Group 4), and normozoospermia (Group 5), and fertile participants were assigned to one of two fertile groups, which include individuals having normozoospermia associated with normal fertility (Fertile Control Group 1) and obstructive azoospermia (Fertile Control Group 2). The characteristics of these groups of individuals are summarized in Table 7. Each individual was karyotyped and tested for microdeletions in YqAZF prior to inclusion in this study.

For the fertile men with obstructive azoospermia (Control Group 2) and infertile men presenting with azoospermia or severe oligozoospermia (Infertile Groups 1a and 1b and 2) frozen or paraffin embedded testis tissue residual to a diagnostic biopsy was used for subsequent PCR and for determination of germ line aneuploidy by fluorescent in situ hybridization (FISH). In some cases, germ cells residual to needle aspiration of the epididymis or testis tubules used for diagnostic purposes and for ICSI were archived for use in this study.

PCR amplification of microsatellite markers from single-sperm PEP products. Single cells were obtained by flow sorting sperm cells or control lymphocytes by fluorescence-activated cell sorting (FACS). DNA was obtained by alkaline lysis of the sorted cells, followed by neutralization. Whole-genome amplification of DNA from single cells was performed using primer-extension pre-amplification (PEP). Microsatellite loci of the PEP DNA were amplified by PCR amplification and the amplification products were separated by capillary electrophoresis on ABI PRISM® 310 or 3100 Genetic Analyzers (Applied Biosystems, Foster City, Calif.).

Small pool PCR (SP-PCR) amplification of microsatellite markers. For some experiments, DNA was purified from whole semen samples and diluted to single or low copy numbers, followed by SP-PCR. Genomic DNA for SP-PCR was extracted from 50 μl of semen using DNA IQ™ System (Catalog Nos. DC6701 and DC6700, Promega Corp.) with the Tissue and Hair Extraction Kit (Catalog No. DC6740, Promega Corp.) and quantified using PicoGreen dsDNA Quantitative Kit (Molecular Probes, Eugene Oregon) following the manufacturer's protocols. Matching blood samples from semen donors were purified using DNA-IQ™ System (Catalog Nos. DC6701 and DC6700, Promega Corp.) which simultaneously quantifies DNA yielding 100 ng at 1 ng/μl. DNA from matching sperm and blood samples were diluted to 1 to 10 genome equivalents (6-60 pg) per PCR reaction and amplified with multiplex sets of fluorescently labeled primers as described below.

The approximate number of genome equivalents was estimated by amplifying increasing amounts (0.1-1 μl) of a 10 pg/μl DNA dilution in a total of 10 PCR reactions, followed by Poisson analysis of the number of reactions positive and negative for a given marker. For each mutation analysis, at least one 96-well plate was used per locus (or multiplex) with each PCR containing 10 genome equivalents (60 pg) of DNA.

Large or small pool PCR amplification of microsatellite markers from testicular tissue. DNA was purified from tissue residual to microsurgical epididymal sperm aspiration or open testicular biopsy of clinically selected men with non-obstructive azoospermia or obstructive azoospermia (control) using the DNA IQ™ System with the Tissue and Hair Extraction Kit (Catalog No. DC6740 from Promega Corp., Madison, Wis.) according to the manufacturer's instructions for subsequent MSI analysis using large or small pool PCR amplification.

PCR amplification and analysis. DNA from blood samples was amplified using 1 ng DNA per PCR reaction following standard protocols described in GenePrint PowerPlex® 16 System and MSI Analysis System Technical Manuals (Promega Corp., Madison, Wis.). For single sperm analysis, 1 ng of PEP DNA from at least 96 samples was amplified by multiplex PCR following the same protocol used with blood samples. DNA for SP-PCR reactions was diluted to 6-60 pg/reaction and at least 30 separate aliquots (small pools) were amplified using 35-40 cycles for each microsatellite multiplex analyzed. Primers for microsatellite markers were from Research Genetics CHLC/Weber Human Screening Set Version 9.0 (Research Genetics, Huntsville, Ala.) or were designed with Oligo Primer Analysis Software version 6.86 (National Biosciences, Plymouth, Minn.). All PCR was performed in ABI GeneAmp® PCR system 9660 or 9700 thermal cyclers.

Amplification products were separated by capillary electrophoresis on ABI PRISM® 310 or 3100 Genetic Analyzers and alleles were sized using ILS-600™ 60-600 bp (Promega Corp., Madison, Wis.) or GeneScan™-2500 55-5117 bp (Applied Biosystems, Foster City, Calif.) as internal lane standards. The appearance of new alleles not present in corresponding somatic cell DNA was scored as a mutation. Germ line specific microsatellite instability was determined by identification of new alleles in sperm DNA that are not present in normal somatic cells from the same individual. Each sample was genotyped by determining allele sizes, and data from different replications was pooled to determine allele number and frequencies for each locus.

Microsatellite instability classification was according to guidelines suggested by the International Workshop on Microsatellite Instability. That is, if more than five markers were used in the panel, tumor samples having ≧30% of loci altered were classified as MSI-high (MSI-H), samples having <30% of loci altered were classified as MSI-low (MSI-L), and samples with no alterations were classified as microsatellite stable (MSS). MMR protein expression in MSI-High and MSI stable tumor samples was evaluated by immunohistochemistry.

Measuring instability in microsatellite or extended mononucleotide repeat loci in samples from azoospermic or severely oligozoospermic men with partial meiotic arrest. Preliminary experiments were conducted to determine the degree of microsatellite instability in DNA from pooled sperm cells and/or DNA from testis biopsies obtained from 25 infertile men, including azoospermic or severely oligozoospermic men, relative to that of DNA from sperm of four fertile men. The DNA was amplified by PCR (35 cycles) in multiplex reactions using fluorescently labeled primer sets and analyzed by capillary electrophoresis on an ABI 3100 instrument. Small pool PCR was performed with MSI Multiplex-1 only by diluting sperm DNA to around 1 to 10 genome equivalents prior to amplification in order to detect new alleles present in less than 10% of cells.

MSI in pooled sperm samples was determined by analyzing the products of multiplex PCR reactions using a number of different microsatellite marker panels including:

(1) MSI Multiplex-1, a marker set optimized for detection of MSI in mismatch repair deficient tumors which contains four mono-nucleotide repeats (BAT-25, BAT-26, MONO-27, and BAT-40) and five tetranucleotide repeat loci (D3S2432, D7S3070, D7S3046, D7S1808 and D10S1426);

(2) MSI Multiplex-2 (MSI Analysis System, Version 1.1, Catalog Nos. MD1641 and 1650, Promega Corp., Madison, Wis.), another marker set optimized for detection of MMR deficient tumors which contains five mononucleotide repeats (BAT-25, BAT-26, NR-21, NR-24, MONO-27) and two pentanucleotide repeat markers (Penta C and Penta D);

(3) PowerPlex® 16 System (Catalog Nos. DC6531 and DC6530, Promega Corp., Madison, Wis.), a multiplex set containing markers with low mutation and stutter rates for use in DNA typing applications that includes thirteen tetra-nucleotide repeats (D18S51, D21 S11, THO1, D3S1358, FGA, D8S1179, CSFlPO, D16S539, D7S820, D13S317, and D5S818), two pentanucleotide repeats (Penta D and Penta E) and a sex determining locus amelogenin; and

(4) PowerPlex®Y System (Catalog Nos. DC6761 and DC6760, Promega Corp., Madison, Wis.), a multiplex of 12 tri-, tetra-, and pentanucleotide repeats on the Y chromosome (DYS391, DYS389I, DYS439, DYS389II, DYS438, DYS437, DYS19, DYS392, DYS393, DYS390, and DYS385a and DYS385b).

In addition to evaluating instability in microsatellite loci, select extended mononucleotide repeat loci were evaluated for instability, including hBAT-51d, hBAT-53c, hBAT-60A, hBAT-62, hBAT-52A, hBAT-59A, hBAT-56a, and hBAT-56b. Table 3 lists each of the extended mononucleotide repeat loci identified in a search of available sequence information.

TABLE 3
Extended Mononucleotide Repeat Loci

| Marker ID | | Accession | | |
|---|---|---|---|---|
| Number | Repeat | Number | Primer Sequence | |
| hBAT-48 | (A)48 | AL162713 | TATAATTAGGTCCCAGATCACTTA | SEQ ID NO:93 |
| hBAT-48 | (A)48 | AL162713 | GGCAATGTTTAAAGACATGGATAC | SEQ ID NO:94 |
| hBAT-49a | (A)49 | AC073648 | AAACACAGTGAGACTCCCTATCTA | SEQ ID NO:95 |
| hBAT-49a | (A)49 | AC073648 | ACAGGACAGAGATGGCACGGACAG | SEQ ID NO:96 |
| hBAT-49b | (A)49 | NT_011757 | CTGCTGTTGCATCGCGGCCCAATG | SEQ ID NO:97 |
| hBAT-49b | (A)49 | NT_011757 | AAGAAGCCCCTCTCCTCCGGTCTC | SEQ ID NO:98 |
| hBAT-50a | (A)50 | NT_011669 | AGGCATGGGCAAGGACTTGATGTC | SEQ ID NO:99 |
| hBAT-50a | (A)50 | NT_011669 | CTGGATGTTAGCCGTTTGTCAGAG | SEQ ID NO:100 |
| hBAT-50b | (A)50 | NT_025441 | GGTTTGCTTGAGGCCAGAACTTCA | SEQ ID NO:101 |
| hBAT-50b | (A)50 | NT_025441 | CTCATAGCAGCCTTAAATTACTGA | SEQ ID NO:102 |
| hBAT-51a | (A)51 | BX908732 | AGCCTGGGCGACAGAGCAAGACTC | SEQ ID NO:103 |
| hBAT-51a | (A)51 | BX908732 | CAAGGGCAGCATCATTATGACAAC | SEQ ID NO:104 |
| hBAT-51b | (A)51 | NT_011630 | TGTGTGCAAATTGTGAGGGAGGTAGGTA | SEQ ID NO:105 |
| hBAT-51b | (A)51 | NT_011630 | AGCGGGGTGCGGTGGCTCATATCT | SEQ ID NO:106 |
| hBAT-51c | (A)51 | NT_011786 | CTGAGGCAGGAGAATGGAGAGTAG | SEQ ID NO:107 |
| hBAT-51c | (A)51 | NT_011786 | CTCTGCTACCCGGGTTCAAACAGT | SEQ ID NO:108 |
| hBAT-51d | (A)51 | NT_011903 | GAGGCTGAGGCAGGAGAATGGCGTGAAC | SEQ ID NO:109 |
| hBAT-51d | (A)51 | NT_011903 | CGCTGACGCAGAACCTGAAATTGTGATT | SEQ ID NO:110 |
| hBAT-51e | (A)51 | NT_025965 | AGGTTGCAGTGAGCCAGGATCATA | SEQ ID NO:111 |
| hBAT-51e | (A)51 | NT_025965 | ATCACATCATCTGTCCCACCTAAC | SEQ ID NO:112 |
| hBAT-51f | (A)51 | NT_079573 | TGGGCGACAGAGCGAGACTCCGTC | SEQ ID NO:113 |
| hBAT-51f | (A)51 | NT_079573 | CAGCGGCCCATAAATTCTATGTTA | SEQ ID NO:114 |
| hBAT-52a | (A)52 | NT_011669 | CTAACTTCCCAGCAACTTCCTTTACACT | SEQ ID NO:115 |
| hBAT-52a | (A)52 | NT_011669 | ATTGGGCAGACACTGAACTAGCTT | SEQ ID NO:116 |
| hBAT-52b | (A)52 | NT_025319 | GGGAGAACCTTGCTGTCTTTCAGATAAT | SEQ ID NO:117 |
| hBAT-52b | (A)52 | NT_025319 | AGGGCTCCTGGAATATGGTTGTAC | SEQ ID NO:118 |
| hBAT-53a | (A)53 | AJ549502 | AACCTCCACCTTCCCAGCTCAAGTGACA | SEQ ID NO:119 |
| hBAT-53a | (A)53 | AJ549502 | GGCGACAGCGAGACTCCGTCTCA | SEQ ID NO:120 |
| hBAT-53b | (A)53 | NT_011875 | CTGAGGCAGGAGAATGGCGTGAAC | SEQ ID NO:121 |
| hBAT-53b | (A)53 | NT_011875 | ATGATGCTGGCCTCATAAAAAGAGTTAG | SEQ ID NO:122 |
| hBAT-53c | (A)53 | NT_011896 | TATCCTAGCTTGGCCTGTTTAAGACC | SEQ ID NO:123 |
| hBAT-53c | (A)53 | NT_011896 | TGAGGCAGGAGAATGGCGTGAA | SEQ ID NO:124 |
| hBAT-54 | (A)54 | NT_077819 | TTTAATATACCTGCTGATCAATGATA | SEQ ID NO:125 |
| hBAT-54 | (A)54 | NT_077819 | GACACATGGGATCATAGCAAA | SEQ ID NO:126 |
| hBAT-55 | (A)55 | NT_028405 | TTGGGCGACAGAGCAAGACGACTC | SEQ ID NO:127 |
| hBAT-55 | (A)55 | NT_028405 | ATTTGGTCAGTGGGGGCTCTGTTAAG | SEQ ID NO:128 |
| hBAT-56a | (A)56 | NT_011726 | TCAGCAGCTGAAAGAAATCTGAGTAC | SEQ ID NO:129 |
| hBAT-56a | (A)56 | NT_011726 | GCGATACCCAAAGTCAATAGTC | SEQ ID NO:130 |
| hBAT-56b | (A)56 | NT_011757 | GAAGCTGCAGTAAGCCGAGATTGT | SEQ ID NO:131 |
| hBAT-56b | (A)56 | NT_011757 | GCGCTCTTAACTCCCATGACATTC | SEQ ID NO:132 |
| hBAT-57 | (A)57 | NT_011875 | AGCCTGGGCGACAGAGCGAGTC | SEQ ID NO:133 |
| hBAT-57 | (A)57 | NT_011875 | CTCGGGGCTCGGGAGATGAGTGA | SEQ ID NO:134 |
| hBAT-59 | (A)59 | AC090424 | CAGCCTAGGTAACAGAGCAAGACCTTTG | SEQ ID NO:135 |
| hBAT-59 | (A)59 | AC090424 | GTTTGCGTGATTTGCGTGGACTT | SEQ ID NO:136 |
| hBAT-59b | (A)59 | NT_010783 | CTCCTGCCTCATCCTCCCGAGTA | SEQ ID NO:137 |
| hBAT-59b | (A)59 | NT_010783 | CCGAGATCACGCCACTGCACTCTA | SEQ ID NO:138 |
| hBAT-60a | (A)60 | NT_008183 | TCTCATTTGAGTGGTGGAAGTGACTGGT | SEQ ID NO:139 |
| hBAT-60a | (A)60 | NT_008183 | TATTCTTTCGGGATGTAATCTCT | SEQ ID NO:140 |
| hBAT-60b | (A)60 | NT_022517 | CCCGTCTCTACTAAAAATACTAAAAC | SEQ ID NO:141 |
| hBAT-60b | (A)60 | NT_022517 | AAACCAACAATAAGGCAACCTCTTAGTC | SEQ ID NO:142 |
| hBAT-60c | (A)60 | NT_023089 | TGCCAGAGTAGGGTGGTCCATGGTACTT | SEQ ID NO:143 |
| hBAT-60c | (A)60 | NT_023089 | GCCCAAAATGTGTTTAGTTAGCTTC | SEQ ID NO:144 |
| hBAT-62 | (A)62 | NT_005120 | AGGCTGAAGCAGGAGAATCACTTAAAAC | SEQ ID NO:145 |
| hBAT-62 | (A)62 | NT_005120 | GCCAAGTGTCGCTTGTAATTCTATT | SEQ ID NO:146 |
| hBAT-63a | (A)63 | NT_009775 | GAATCTTGTTTCGGCCTTTGACCTTA | SEQ ID NO:147 |
| hBAT-63a | (A)63 | NT_009775 | CGAGATCACGCCACCGCACTCTAGC | SEQ ID NO:148 |
| hBAT-63b | (A)63 | NT_022184 | AAATCTACCCAGCTCTGTAACGAGAGA | SEQ ID NO:149 |
| hBAT-63b | (A)63 | NT_022184 | AAGCTCTGTTTGGCAAGTGTTAATTGTA | SEQ ID NO:150 |
| hBAT-68a | (A)68 | NT_016354 | TTGGAATGTATTCTCTGGGTTTGGCAGT | SEQ ID NO:151 |
| hBAT-68a | (A)68 | NT_016354 | TTCAGGAGGCTGAGGTGGGAGGATTGT | SEQ ID NO:152 |
| hBAT-68b | (A)68 | NT_079574 | ACCTAGGCAATACCATCTAAGA | SEQ ID NO:153 |
| hBAT-68b | (A)68 | NT_079574 | GTTGCCTGTTCACTCTGATAGTCT | SEQ ID NO:154 |
| hBAT-69 | (A)69 | NT_032977 | AGCCTGGGTGACAGAGCGAGACT | SEQ ID NO:155 |
| hBAT-69 | (A)69 | NT_032977 | TTAGAGTTATTTGTTGGGATGAGAATCT | SEQ ID NO:156 |
| hBAT-72 | (A)72 | NT_037623 | CTGGGCGACAGAGCGAGACTCC | SEQ ID NO:157 |
| hBAT-72 | (A)72 | NT_037623 | TCTCCTGCCTTAGCCTCCCGAGTAGC | SEQ ID NO:158 |
| hBAT-73 | (A)73 | NT_079596 | TCCTCTCCCTAAAAAGCTCCCCCTAAG | SEQ ID NO:159 |
| hBAT-73 | (A)73 | NT_079596 | AGGTCAAGGCTGCGGTAAGCTGTGATCG | SEQ ID NO:160 |
| hBAT-79 | (A)79 | NT_010194 | TCCCCACTTTGTCCTGCACACTCCTACC | SEQ ID NO:161 |
| hBAT-79 | (A)79 | NT_010194 | GGGCGACAGAGCGAGACTCCGTC | SEQ ID NO:162 |
| hBAT-83 | (A)79 | NT_007422 | AAGATTTAATAGACATGCGCAGAACACT | SEQ ID NO:163 |
| hBAT-83 | (A)83 | NT_007422 | CCAGCCTGGGCAAAAGAGCAAGT | SEQ ID NO:164 |
| hBAT-90 | (A)90 | NT_029419 | ACAAACATGAAAAGGCAAATGATAGAAC | SEQ ID NO:165 |
| hBAT-90 | (A)90 | NT_029419 | AGAGGTTGCAGTGAGCCAAGATTGTAG | SEQ ID NO:166 |

Electropherograms were evaluated by determining the number and size of amplification products for each locus. The presence of more than two alleles at a locus was scored as MSI (+).

Results from large pool PCR experiments are given in Table 4 along with the phenotypes and summaries of the details about subjects included in preliminary studies. Of 25 tested samples from infertile men, two, designated I-14 and I-30, displayed relatively high levels of MSI (29% and 47%, respectively), which is comparable to MSI seen in tumor tissues with a defect in mismatch repair. None of the samples from fertile men showed instability.

TABLE 4
Frequency of MSI in sperm DNA from infertile and fertile men.

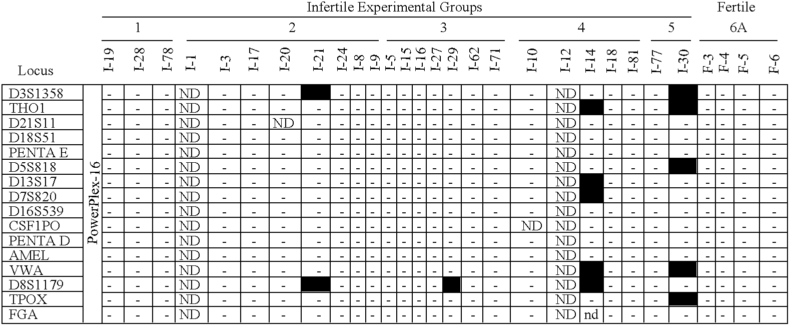

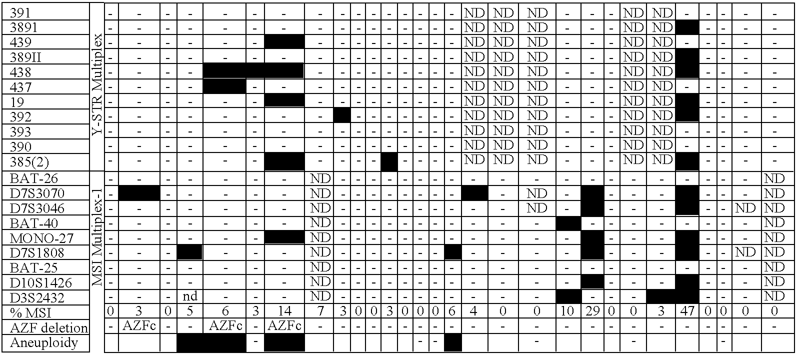

NCI guidelines for MSI determination require alteration in greater than 30% of the markers to be considered diagnostic of MMR dysfunction. Typically, instability is observed in greater than 70% of MSI Multiplex markers in colorectal tumors that lack expression of MSH2 or MLH1 mismatch repair proteins. However, high rates of MSI in MMR deficient tumors are likely due to clonal evolution of tumors that allows accumulation of multiple changes in repeat loci along with larger shifts in number of repeat units. NCI guidelines were used to determine if germ line genomic instability is analogous to MSI in the MMR deficient somatic cell tumor. To avoid employing a selection process that is too stringent for germ line GI in the initial studies, microsatellite markers that show alterations in 20% to 30% of alleles across germ line samples were retained for further evaluation in loci panels for comparison to other more sensitive loci.

Using samples containing large numbers of cells (i.e., pooled DNA) has the disadvantage of not allowing detection of new alleles due to masking when the new alleles occur in less than 10% of the total population. In order to accurately detect low frequency MSI in sperm samples and as a control, two methods were used to permit evaluation of a single cell or a small number of cells. Sperm were flow sorted for single cell analysis and amplified with NCI panel markers D2S123, D5S346, Dl7S250 and MYCL1. In addition, MSI of flow sorted sperm were evaluated using Y-chromosome loci and select mononucleotide and dinucleotide repeats. DNA from lymphocytes was amplified in multiplex reactions as a control. Non-constitutive alleles that arise as a result of MSI could be identified by comparing results obtained for single cell sperm cells with those obtained for control somatic cells (lymphocytes). New alleles occurred at an overall frequency of 28% for D5S346, 29% for D17S250, 32% for D2S123 and 39% for MYCL1. This was a considerably higher frequency than observed in total pooled sperm sample analysis.

Small-pool PCR was also used to detect MSI in samples from infertile men using Multiplex-1, MSI Multiplex-2, and PowerPlex®Y markers (Table 5). For each sample, pooled sperm DNA was diluted to 1-10 genome equivalents and then amplified with multiplex PCR. SP-PCR products were resolved by capillary electrophoresis using a sequencing polymer that gives 1-bp resolution of DNA fragments. The SP-PCR data revealed MSI in at least one locus in all but one of the infertile samples (Table 5). No MSI was seen in matched blood samples from these individuals. Likewise, none of the fertile germ line and soma samples tested displayed MSI, indicating that mutations observed in infertile samples were not due to PCR artifacts. Both single sperm and SP-PCR revealed cryptic mutations and presence of MSI not normally detectable with standard large pool PCR.

TABLE 5
Frequency of MSI in sperm DNA from infertile men using small pool PCR

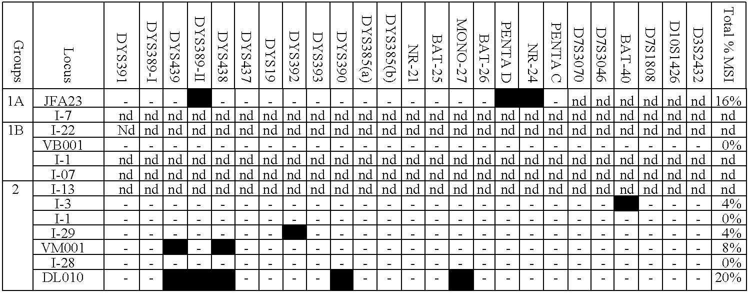

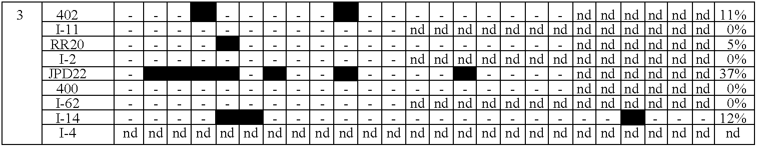

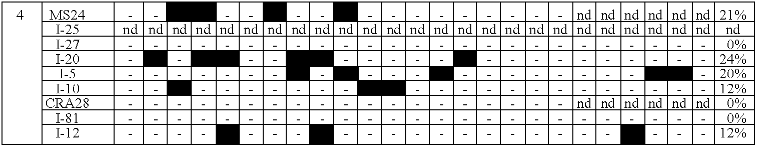

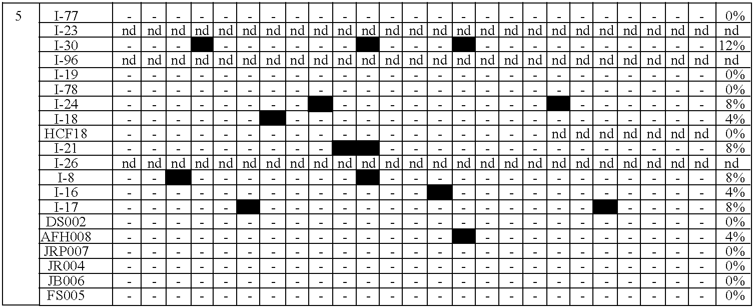

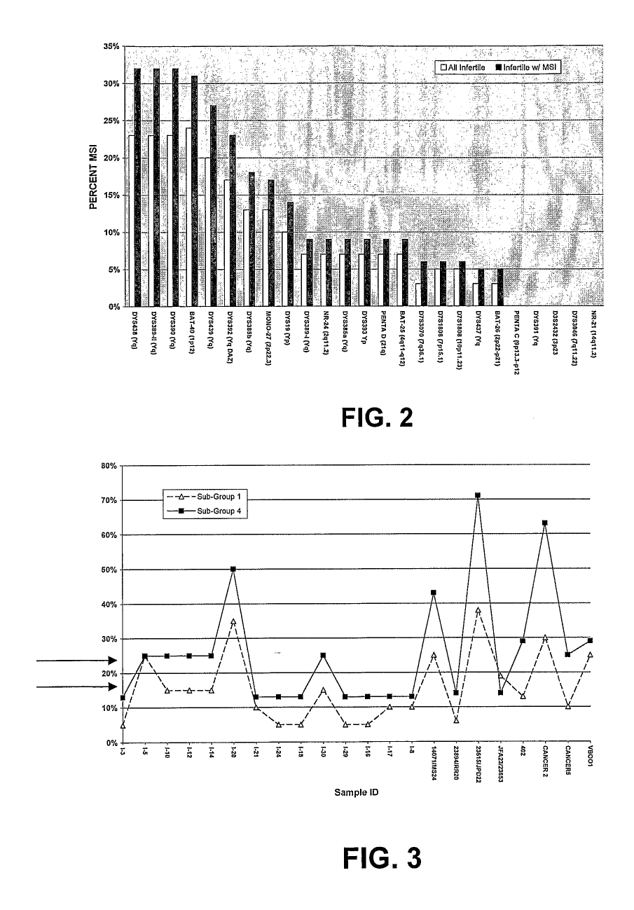

To further evaluate whether repetitive DNA sequences are preferentially unstable in the sperm cells or testis of infertile men, and that the susceptibility of an individual locus to instability varies according to its DNA sequence and its chromosomal location, 25 loci distributed across autosomes and the Y chromosome were combined in five multiplex reactions to evaluate two populations of infertile men (i.e., 30 men selected on the basis of spermatogenic arrest and 22 men selected on the basis of having germ line MSI in at least one locus). As an internal amplification control, two of the STR multiplexes were constructed with intentional redundancy of three loci. This approach streamlined the reactions and improved assay sensitivity. The distribution of the loci and mutation rates are shown in FIG. 2, with white bars denoting the frequency of MSI for each locus in men clinically selected on the basis of spermatogenic arrest, and black bars indicating frequency of MSI for each locus in men selected on the basis of germ line instability in at least one locus.

Microsatellite loci were amplified from DNA from sperm or testis biopsy and blood from 22 infertile men with germ line instability in at least one locus in large pool and/or small pool reactions with a minimum of from 16 to 80 replicates per data locus. Average replicates per pool of germ line and soma per locus was 45. Similar numbers of replicate amplifications of blood samples were studied as controls for each sperm sample. As a control, DNA from sperm and blood samples from 6 fertile sperm donors was amplified. No mutations were noted in the soma from infertile or fertile men, and no mutations were found in the sperm of fertile men. The mutation frequencies for loci in infertile males are summarized in FIG. 3. The solid line plots the percent MSI for the eight loci exhibiting the greatest sensitivity according to the results summarized in FIG. 2 (i.e., DYS438, DYS389-II, DYS390, BAT-40, DYS439, DYS392, DYS385b, and MONO-27), and the broken line indicates the percent MSI for a set of 19 loci (i.e., DYS438, DYS389-II, DYS390, BAT-40, DYS439, DYS392, DYS385b, MONO-27, DYS19, DYS389-1, NR-24, DYS385a, DYS393, PENTA D, BAT-25, D7S3070, DS 1808, DYS437,and BAT-26).

### D. Evaluation of Sensitivity of Y Chromosome Microsatellite Loci in MMR Deficient Tumors.

The stabilities of 12 select Y-chromosome microsatellites were evaluated in four MMR deficient colon cancer tumors and 15 MMR proficient colon cancer tumors in large pool PCR experiments. The MMR status of each of the tumors was confirmed by immunohistochemistry of proteins associated with MMR. The data is summarized in Table 6. All but one of the Y-chromosome markers tested exhibited some level of instability in one or more of the MMR deficient tumors, indicating susceptibility of these markers to alterations in the absence of DNA mismatch repair. In contrast, the Y-STR markers were nearly stable in mismatch repair proficient tumors, which indicates that these markers are susceptible to mutations in mismatch repair defective cells, suggesting that the high levels of instability of these markers in sperm samples from infertile men may be related to loss of mismatch repair.

TABLE 6

| MMR deficient | | | | | MMR proficient | | | | | | | | | | | | | | |
|---|---|---|---|---|---|---|---|---|---|---|---|---|---|---|---|---|---|---|---|
| DYS391 | 0% | 0% | 100% | 33% | 0% | 0% | 100% | 0% | 0% | 0% | 0% | 0% | 0% | 0% | 0% | 0% | 0% | 0% | 7% |
| DYS389 I | 100% | 0% | 100% | 67% | 0% | 0% | 0% | 0% | 0% | 0% | 0% | 0% | 0% | 0% | 0% | 0% | 0% | 0% | 0% |
| DYS438 | 100% | 100% | 100% | 100% | 0% | 0% | 0% | 0% | 0% | 0% | 0% | 0% | 0% | 0% | 0% | 0% | 0% | 0% | 0% |
| DYS389 II | 100% | 0% | 100% | 67% | 0% | 0% | 0% | 0% | 0% | 0% | 0% | 0% | 0% | 0% | 0% | 0% | 0% | 0% | 0% |
| DYS438 | 100% | 100% | 0% | 67% | 0% | 0% | 0% | 0% | 0% | 0% | 0% | 0% | 0% | 0% | 0% | 0% | 0% | 0% | 0% |
| DYS437 | 0% | 0% | 100% | 33% | 0% | 0% | 0% | 0% | 0% | 0% | 0% | 0% | 0% | 0% | 0% | 0% | 0% | 0% | 0% |
| DYS19 | 0% | 100% | 100% | 47% | 0% | 0% | 0% | 0% | 0% | 0% | 0% | 0% | 0% | 0% | 0% | 0% | 0% | 0% | 0% |
| DYS392 | 100% | 0% | 100% | 67% | 0% | 0% | 0% | 0% | 0% | 0% | 0% | 0% | 0% | 0% | 0% | 0% | 0% | 0% | 0% |
| DYS393 | 0% | 100% | 100% | 67% | 0% | 0% | 0% | 0% | 0% | 0% | 0% | 0% | 0% | 0% | 0% | 0% | 0% | 0% | 0% |
| DYS390 | 100% | 0% | 0% | 33% | 100% | 0% | 0% | 0% | 0% | 0% | 0% | 0% | 0% | 0% | 0% | 0% | 0% | 0% | 7% |
| DYS385 (a) | 100% | 0% | 0% | 33% | 0% | 0% | 0% | 0% | 0% | 0% | 0% | 0% | 0% | 0% | 0% | 0% | 0% | 0% | 0% |
| DYS385 (b) | 0% | 0% | 0% | 0% | 0% | 0% | 0% | 0% | 0% | 0% | 0% | 0% | 0% | 0% | 0% | 0% | 0% | 0% | 0% |
| Total | 58% | 33% | 67% | 33% | 3% | 0% | 8% | 0% | 0% | 0% | 0% | 0% | 0% | 0% | 0% | 0% | 0% | 0% | 1% |

### E. Detection of a Testicular Mutator Phenotype.

Because some MMR proteins function in meiosis and, in soma cells, in DNA repair, it may be that both MSI and chromosomal instability are hallmarks of the germ line specific mutator phenotype. This is in contrast to tumors, which exhibit MSI or chromosomal instability, but not both. Endpoints included alterations at selected STR loci from across the genome (defined above) and measurements of germ line aneuploidy by FISH.

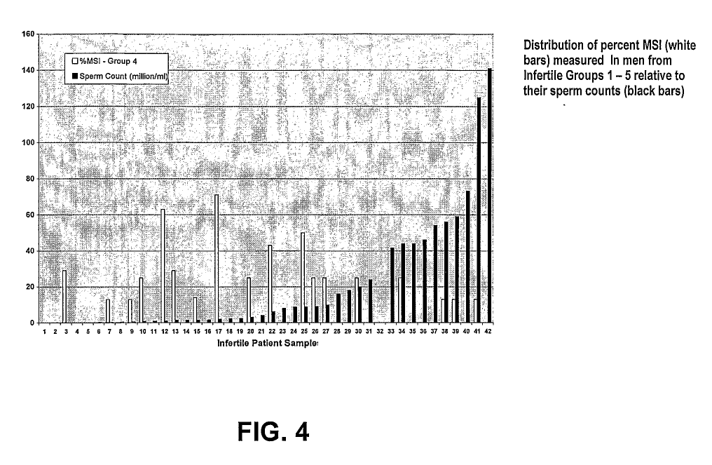

Detection of germ line specific genomic instability in infertile men. In preliminary experiments, germ line GI sensitive microsatellite loci described above were used to measure instability in the germ line and soma of expanded populations of infertile (n=38) and fertile (n=11) men using small pool PCR in parallel with single cell PCR on flow sorted cells. The infertile population was divided into 5 groups and the fertile population was divided into 2 groups (Table 7). Ages ranged from 26 to 59 in the fertile population and from 22 to 71 in the infertile population. Individuals included in this study were from a broad range of ethnic groups derived from infertility centers in Columbia, Panama, New York and Wisconsin. For small pool experiments we used up to 40 markers in up to 80 small pool replicates for the germ line and for the soma. DNA was purified from the germ line and soma of each man using DNA IQ (Promega, Corp. Madison, Wis.). For samples containing mature or immature germ cells, Tomah was used as a detergent for homogeneous lysis with PicoGreen. Concentration was determined using PicoGreen dsDNA Quantitation Kit (Molecular Probes, Eugene, Oreg.). DNA was diluted to 1-2 molecules, amplified in 96 well plates with 16 negative (blank) controls, and the amplification products were separated and detected by capillary electrophoresis using an Applied Biosystems 3100 Genetic Analyzer. All preamplification steps were performed in a sterile laminar flow hood to avoid PCR contamination. The data was analyzed using AB GeneScan and Genotyper Software Analysis packages to identify the presence of microsatellite mutations. Calculations of mutations causing new alleles and MSI employed a conservative approach. Signals indicative of PCR artifact, dye interference, or stutter were not scored. Germ line genomic instability in all subjects was scored according to the protocol adopted by the NCI for diagnosing MSI in MMR deficient tumors. Data is summarized in Table 8. FIG. 4 shows distribution of percent MSI (white bars) and sperm cell concentrations (black bars), and reflects that high percent MSI values coincide with low sperm cell concentrations. The negative correlation between MSI and sperm count is significant (p<0.05). In addition, percent MSI correlates with increased age and abnormal sperm head morphology and inversely correlated with motility.

| | | | | | Number | | |
|---|---|---|---|---|---|---|---|
| | | | Number | Number | Samples | | Samples |
| | | Sperm | Samples | Samples | to be | | Types |
| Group | Phenotype | Count | Tested | Archived | Collected | Range of pathologies | Collected |
| Infertile | Non-obstructive | No | 2 | 9 | 14 | No germ cells present in the | B/T |
| Group 1a | Azoosparmia | sperm | | | | [symbol] coupled with | |
| | | | | | | histological demonstration of | |
| | | | | | | no germ cells apparently | |
| | | | | | | present [symbol] the [symbol] S [symbol] | |
| | | | | | | Cells Only (SCO), | |
| | | | | | | [symbol] Azoosparmia | |
| Infertile | Non-obstructive | No | 4 | 9 | 12 | Meiotic/spermatoganic | B/S/T |
| Group 1b | Azoosparmia | sperm | | | | arrest/Post- | |
| | | | | | | Meiotic/spermatoganic arrest | |
| | | | | | | m [symbol] | |
| | | | | | | Partial [symbol] | |
| | | | | | | spermatoganic | |
| | | | | | | [symbol] | |
| Infertile | Severe | ≦1 × 10⁵ | 7 | 26 | 15 | Partial maturation arrest | B/S/T |
| Group 2 | Oligozoospermia | sperm/ml | | | | Idiapa [symbol] Partial | |
| | | | | | | [symbol] arrest | |
| | | | | | | hyposperralogenesisT [symbol] | |
| | | | | | | zoospermia in some ca [symbol] | |
| Infertile | Moderate | [symbol] 1 × 10⁴ | 9 | 19 | 22 | Partial maturation arrest | B/S |
| Group 3 | Oligozoospermia | to 5 × 10 [symbol] | | | | [symbol] | |
| | | sperm/ml | | | | | |
| Infertile | Med | >5 × 10 [symbol] to | 9 | 22 | 19 | Partial maturation arrest | B/S |
| Group 4 | Oligozoospermia | 20 × 10 [symbol] | | | | [symbol] | |
| | | sperm/ml | | | | | |
| Infertile | Normozoospermia | >20 × 10 [symbol] | 20 | 30 | 0 | NomozoospermiaVa [symbol] | B/S |
| Group 5 | | | | | | <5% normal morplio [symbol] | |
| | | | | | | [symbol] monted heads, other | |
| | | | | | | head abria [symbol] , | |
| Fertile | Normozoospermia | >20 × 10 [symbol] | 10 | 6 | 7 | Normal spermatogeness and | B/S |
| CONTROL | Associated with | | | | | Normal sperm [symbol] | |
| Group 1 | Normal Fertility | | | | | | |
| Fertile | Obstractive | No | 1 | 9 | 15 | CBAVDINormal | B/S/T |
| CONTROL | Azoospermia | sperm in | | | | spermalogenesis | |
| Group 2 | | epaculass | | | | | |
| TOTALS | | | 62 | 134 | 104 | | |

[symbol]

indicates data missing or illegible when filed

TABLE 8

| | | | | Normal | MSI % | | | |
|---|---|---|---|---|---|---|---|---|
| Infertile | | Age | Count | Morph | SP-PCR | Deletion | Germ line | |
| Groups | Sample ID | (yrs) | (10⁶/ml) | (%) | Group_4 | in AZF | aneuploidy | SCSA % DFI |
| 1a | JFA23/23653 | 44 | 0 | 0 | 14 | no | tbd - biopsy collected | insufficient sperm |
| 1a | I-7 | 44 | 0 | 0 | tbd | no | nd | insufficient sperm |
| 1b | I-22 | 41 | 0 | 0 | tbd | no | nd | insufficient sperm |
| 1b | VB001* | 38 | 0.004 | 0 | 29 | no | tbd - biopsy collected | insufficient sperm |
| 1b | I-1 | 43 | 0 | 0 | tbd | AZFc | yes | insufficient sperm |
| 1b | I-97 | 37 | 0.0002 | 0 | tbd | no | tbd - biopsy collected | insufficient sperm |
| 2 | I-13 | 43 | 0.2 | 5 | tbd | no | tbd - biopsy collected | insufficient sperm |
| 2 | I-3 | 41 | 0.2 | 0 | 13 | AZFc | yes | insufficient sperm |
| 2 | I-1 | 33 | 0.4 | 0 | 0 | no | yes | insufficient sperm |
| 2 | I-29 | 59 | 0.5 | 0 | 13 | no | tbd - biopsy collected | insufficient sperm |
| 2 | VM001* | 40 | 0.8 | 0 | 25 | no | tbd - biopsy collected | insufficient sperm |
| 2 | I-28 | 45 | 0.9 | 0 | 0 | azfc | tbd - biopsy collected | insufficient sperm |
| 2 | DL010* | 40 | 1 | 0 | 63 | no | tbd - biopsy collected | insufficient sperm |
| 3 | 402 | 57 | 1.4 | 3 | 29 | AZFc | tbd - sperm FISH | insufficient sperm |
| 3 | I-11 | 49 | 1.5 | 0 | tbd | no | tbd - sperm FISH | insufficient sperm |
| 3 | 23894/RR20 | 33 | 1.5 | 0 | 14 | no | no | insufficient sperm |
| 3 | I-2 | 47 | 1.6 | 5 | tbd | no | tbd - sperm FISH | 49.8 |
| 3 | 23615/JPD22 | 53 | 2 | 0 | 71 | no | tbd - sperm FISH | not applicable |
| 3 | 400 | 47 | 2.3 | 48 | 0 | no | no | not applicable |
| 3 | I-62 | 42 | 2.4 | 2 | 0 | no | no | 64.9 |
| 3 | I-14 | 53 | 3 | 1 | 25 | no | yes | 31.7 |
| 3 | I-4 | 47 | 4 | 0 | tbd | no | tbd - sperm FISH | 40.4 |
| 4 | 14071/MS24 | 39 | 6.1 | 0 | 43 | no | no | 47.6 |
| 4 | I-25 | 57 | 8 | 0 | tbd | no | nd | 33.9 |
| 4 | I-27 | 41 | 8.7 | 0 | 0 | no | no | 32.1 |
| 4 | I-20 | 51 | 8.8 | 2 | 50 | AZFc | yes | 69.1 |
| 4 | I-5 | 54 | 9 | 0 | 25 | no | nd | 40.3 |
| 4 | I-10 | 34 | 9.8 | 3 | 25 | no | no | 18.4 |
| 4 | 24009/CRA28 | 37 | 16 | 0 | 0 | no | no | 18.6 |
| 4 | I-81 | 50 | 18 | 1 | 0 | no | no | 17.4 |
| 4 | I-12 | 54 | 20 | 4 | 25 | no | yes | 27.8 |
| 5 | I-77 | 53 | 24 | 2 | 0 | no | nd | 30.2 |
| 5 | I-23 | 49 | 41.6 | 5 | tbd | no | nd | 33.5 |
| 5 | I-30 | 37 | 44 | 0 | 25 | no | yes | 10 |
| 5 | I-98 | 49 | 44 | 0 | tbd | no | nd | 23.5 |
| 5 | I-19 | 40 | 46 | 0 | 0 | no | nd | 30.1 |
| 5 | I-78 | 47 | 54 | 4 | 0 | no | nd | 45.7 |
| 5 | I-24 | 45 | 56 | 7 | 13 | no | nd | 5 |
| 5 | I-18 | 44 | 59 | 3 | 13 | no | nd | 26 |
| 5 | 23936/HCF18 | 40 | 73.1 | 0 | 0 | no | yes | 18.9 |
| 5 | I-21 | 56 | 125 | 2 | 13 | no | no | 5.7 |
| 5 | I-26 | 47 | 141 | 9 | tbd | no | nd | 8.3 |
| 5 | I-8 | 29 | 80 | 66 | 13 | no | nd | 21.6 |
| 5 | I-16 | 40 | 30 | 71 | 13 | no | no | 31.2 |
| 5 | I-17 | 32 | 190 | 6 | 13 | no | no | 30.1 |
| 5 | DS002* | 31 | 29.5 | 20 | 0 | no | nd | nd |
| 5 | AFH008** | 27 | 187 | 15 | 0 | no | yes | nd |
| 5 | JRP007* | 34 | 23.9 | 10 | 0 | no | nd | nd |
| 5 | JR004** | 26 | 56 | 7 | 0 | no | yes | nd |
| 5 | JB006* | 38 | 89 | 15 | 0 | no | yes | nd |
| 5 | FS005*** | 36 | 113 | 15 | 0 | no | nd | nd |
| Fertile 1 | F-1-1 | 29 | 251.5 | 54 | 0 | no | tbd - sperm FISH | nd |
| Fertile 1 | F-1-2 | 37 | 68.2 | 63 | 0 | no | tbd - sperm FISH | nd |
| Fertile 1 | F-1-3 | 41 | 251.5 | 54 | 0 | no | no | nd |
| Fertile 1 | F-1-4 | 22 | 187.2 | 58 | 0 | no | tbd - sperm FISH | nd |
| Fertile 1 | F-1-5 | 45 | 132 | 85 | 0 | no | tbd - sperm FISH | nd |
| Fertile 1 | F-1-66 | 66 | 193.3 | 57 | 0 | no | tbd - sperm FISH | 18.3 |
| Fertile 1 | F-1--7 | 35 | 154.5 | 61 | 0 | no | tbd - sperm FISH | nd |
| Fertile 1 | F-1-9 | 39 | 106 | 63 | 0 | no | tbd - sperm FISH | 18.4 |
| Fertile 1 | F-1-15 | 43 | 44 | 70 | 0 | no | tbd - sperm FISH | Nd |
| Fertile 1 | F-1-71 | 71 | 190 | 65 | 0 | no | tbd - sperm FISH | 19.8 |
| Fertile 1 | F-1-GA | 33 | 87 | 72 | tbd | no | tbd - sperm FISH | 19.5 |

In the clinically selected infertile men, 4 individuals, namely, MS-24, I-20 (Infertile Group 4), JDP-22 (Infertile Group 3) and DL010-(Infertile Group 2) were MSI-H (MSI≧30%). Interestingly, DL-010 was diagnosed with severe oligoasthenoteratozoospermia more than a decade ago and has two brothers with a similar testicular phenotype. Conception of his only child was facilitated through ICSI three years ago, when several ejaculates and needle aspirations were collected and banked. In 2004, DL-010 presented with seminoma and is now beginning his treatment. The germ line instability of DL-010 increased over time from an initial value of 43% for a sample collected in 2001 to 71% in a sample collected in 2004. No mutations were detected in the soma of any of the men tested.

Though the NCI does not have an intermediate MSI category, individuals in this study having germ line GI in the 20-29% range were designated MSI-Intermediate. The MSI-I group includes 11 men, including one from Group 1a, one from Group 1b, one from Group 2, two from Group 3, three from Group 4 and three from Group 5. The germ line MSI in I-14 from Group 5 was detected in early experiments in large and small pool experiments. Of concern was the comparatively high instability in the earliest large pool experiments in this man. Two men in the MSI-I group achieved pregnancies with ICSI during the last few years, but have since been diagnosed with seminoma.

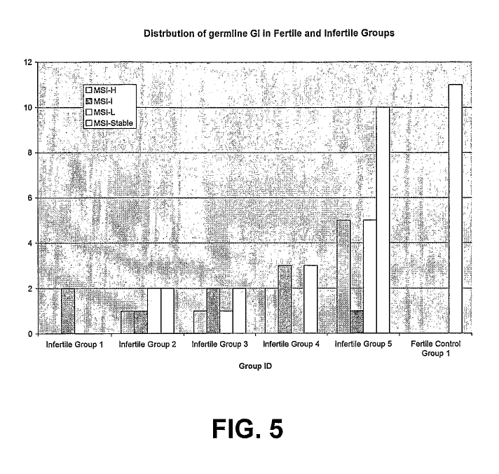

Seven men distributed across Infertile Groups 2-5 are categorized as MSI-Low (MSI-L), with germ line mutations in 5%-19% of tested loci. The remainder of the infertile men studied demonstrated stability in their germ lines equivalent to the soma of both the infertile and fertile men (0% MSI). The germ lines of the fertile males studied to date were similarly stable. FIG. 5 summarizes the distribution of GI in sperm or testicular samples of infertile men across five infertile groups, relative to that of the fertile group.

It is expected that BAT53c and other BATs on either the X or Y chromosome and BATs having at least 38 A's or ROS sensitive markers will also be found to be unstable in the germ line of infertile men at risk of developing seminoma.

Measuring aneuploidy by FISH in age stratified men with spermatogenic arrest. To evaluate chromosomal instability, germ line aneuploidy was determined by FISH for select individuals across both Fertile and Infertile Groups in parallel to MSI experiments described above. To date, 21 men from Infertile Groups 1-5 and one man from fertile control Group 1 have been evaluated. Aspirated or ejaculated sperm samples were thawed as required and washed and slides were prepared according to methods described in McInnes et al. (Hum Reprod 1998; 13:2787-2790), which is incorporated herein by reference. Sperm nuclei were decondensed, rinsed, and air-dried. Fluorescently labeled centromeric probes to Chromosomes X, Y, 18, and 21 were hybridized overnight to sperm according to the recommended protocol for directly labeled probes (Vysis, Inc. Downers Grove, Ill.). After post-hybridization washes, slides were counterstained with DAPI. Only sperm with hybridization to at least 2 of 4 chromosomes were scored to avoid technical failure and artifact. Sperm were scored as haploid, nullisomy or disomy. Results of this experiment were valuable in defining parameters that differentiate between GI associated with chromosomal instability or MSI or in the germ line, perhaps both.

Genomic instability and the Y-chromosome. Repetitive motifs that flank functional genes occur throughout the genome and have been associated with aberrant recombination events that are correlated with a variety of diseases. If a Y intra-chromosomal recombination event occurs in a region containing genes of functional importance, such as RBM and DAZ, the result can be a deletion involving a whole region and subsequent loss of spermatogenesis and fertility. Because of the relatively high frequency of large deletions in the palindromic rich AZF region of Yq in azoospermic and severely oligozoospermic men, the integrity of Yq was evaluated for all samples prior to inclusion in this study. Several of the most sensitive STRs are linked to Yq, just below the centromere and proximal to the region that is most commonly involved in microdeletion in AZF. Five of the 52 men in Infertile Groups 1-5 had deletions that removed the DAZ gene cluster (AZFc) whereas no Yq deletions were detected in 12 similarly screened men with normal spermatogenesis in Fertile Control Groups 1 and 2. Each of the 52 infertile men were also karyotyped as normal 46,XY in peripheral blood lymphocytes by the referring laboratories.

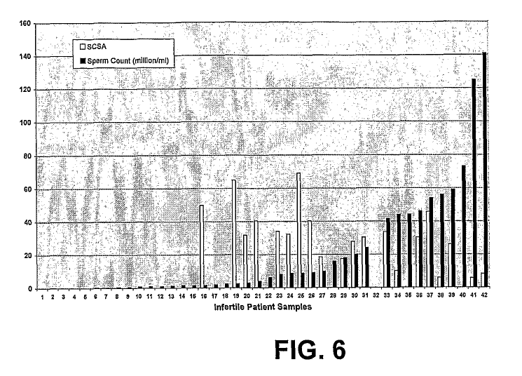

Strand breaks as measured by sperm chromatin structure assay and germ line specific STR instability. Chromatin breaks in 28 infertile men were evaluated using the sperm chromatin structure assay (SCSA). Abnormal SCSA is indicative of DNA strand breaks and is associated with elevated germ line aneuploidy, failed fertilization, and increased miscarriage. The data are summarized in Table 8 as percent total chromatin breaks or fragmentation. In addition, the distribution of percent DFI (DNA Fragmentation Index) (white bars) are shown relative to sperm count (black bars) in FIG. 6. Generally, those individuals with elevated MSI have the most fragmented chromatin as measured by SCSA. Unfortunately, it is not possible to perform SCSA on men with sperm counts below about 2 million. These experiments suggest a positive correlation between elevated percent fragmented sperm chromatin, a marker of GI in sperm, and elevated percent MSI only in Infertile Group 4 (p=0.03) using Pearson Correlation Coefficient. There was a negative correlation across all Infertile Groups tested between elevated percent fragmented sperm chromatin and sperm count or sperm motility (p=0.03 and p=0.004, respectively).

### F. Detection of Genomic Instability in Pluripotent Cells or Stem Cells

Cultured stem cells or pluripotent cells may accumulate mutations while being serially passaged in culture. The presence of mutations and rates of mutation will need to be assessed for these cells to be useful in treating or alleviating diseases. The present invention may be used to assess the accumulation of mutations while in culture by measuring microsatellite instability.

After the stem cells or pluripotent cells are cultured or when these cells are differentiated in culture, and prior to analysis or use of these cells the microsatellite stability will be assessed. DNA will be isolated from the differentiated or cultured stem cells or pluripotent cells by standard techniques. The DNA will be amplified following standard PCR protocols as described earlier. The microsatellite loci may be amplified using the primer sets described in the earlier Examples. Alternatively, PCR primers to any microsatellite loci may be designed using available sequence information and software for designing oligonucleotide primers, such as Oligo Primer Analysis Software version 6.86 (National Biosciences, Plymouth, Minn.).

The amplification products will be separated by capillary electrophoresis on an ABI PRISM® 310 or 3100 Genetic Analyzers and alleles will be sized using ILS-600™ 60-600 bp (Promega) or GeneScan™-2500 55-5117 bp (Applied Biosystems) as internal lane standards. The expected size of the amplification products will be determined by comparing the amplification product from the cultured stem or pluripotent cells to matched amplification products from control DNA. The control DNA may be derived from an earlier or initial sample obtained prior to repeated in vitro passaging or prior to in vitro differentiation or treatment of the cultured stem or pluripotent cells. The expected size of the amplification product could also be determined by a pedigree analysis or comparison to the population if a particular microsatellite locus is monomorphic or quasi-monomorphic in the population.

The appearance of new alleles not present in control DNA samples or not similar to the expected size of the amplification product will be scored as mutations. Microsatellite instability will be determined by identification of new alleles in cultured stem cell or pluripotent cell DNA that are not expected.

A listing of loci suitable for use in the methods of the invention is provided in Table 9. Each locus may be evaluated for mutations either individually or in combination with other loci. To practice the method of the invention, one may conveniently select individual loci or groups of from 2 to 81 loci from the loci listed in Table 9 to be amplified and evaluated for mutations according to the method of the invention. The methods of the invention are not limited to those loci disclosed and can be practiced with any other extended mononucleotide repeat or Y-chromosome short tandem repeat loci.

TABLE 9

Amelogenin
BAT-25
BAT-26
BAT-40
BAT53c
CSF1PO
D10S1426
D13S17
D13S317
D16S539
D17S250
D18S51
D21S11
D2S123
D3S1358
D3S2432
D5S346
D5S818
D7S1808
D7S3046
D7S3070
D7S820
D8S1179
DS1808
DYS19
DYS385a
DYS385b
DYS389-I
DYS389-II
DYS390
DYS391
DYS392
DYS393
DYS434
DYS435
DYS436
DYS437
DYS438
DYS439
DYS446
DYS447
DYS448
DYS449
DYS452
DYS453
DYS454
DYS455
DYS456
DYS458
DYS459a
DYS459b
DYS460
DYS461
DYS462
DYS463
DYS464a
DYS464b
DYS464c
DYS464d
DYS488
FGA
hBAT-51d
hBAT-52a
hBAT-53c
hBAT-56a
hBAT-56b
hBAT59a
hBAT-60a
hBAT-60b
hBAT-62
MONO-27
MYCL1
NR-21
NR-24
Penta B
Penta C
Penta D
Penta E
TH01
TPOX
vWA

1. A method for detecting genomic instability in a germ cell comprising:
(a) obtaining a first DNA sample from at least one germ cell, the first DNA sample comprising at least one microsatellite locus selected from the group consisting of: Y chromosome microsatellite loci; extended mononucleotide repeat loci having at least 41 repeats; and A-rich short tandem repeats having repeating units selected from the group consisting of AAAAG, AAAAC, and AAAAT;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the amplification product being indicative of genomic instability.

2. The method of claim 1, wherein the germ cell is a sperm cell.

3-6. (canceled)

7. The method of claim 1, wherein DNA is isolated from more than one germ cell.

8. The method of claim 5, wherein more than one amplification product per locus is indicative of genomic instability.

9. The method of claim 7, wherein the production of more than two amplification products per locus is indicative of genomic instability.

10. (canceled)

11. The method of claim 1, wherein the at least one microsatellite locus comprises at least one Y chromosome microsatellite locus selected from the group consisting of mononucleotide repeat loci, dinucleotide repeat loci, trinucleotide repeat loci, tetranucleotide repeat loci, and pentanucleotide repeat loci.

12-19. (canceled)

20. The method of claim 1, wherein genomic instability is indicative of infertility.

21. The method of claim 1, wherein the expected size of the amplification product is assessed by a method comprising:
(e) obtaining a second DNA sample from at least one control cell, the second DNA sample comprising the at least one microsatellite locus;
(f) contacting the second DNA sample with the first and second primers of step (b) under conditions that allow amplification of the at least one microsatellite locus to form a second amplification product; and
(g) determining the size of the second amplification product, wherein the size of the second amplification product is the expected size of the amplification product of step (d).

22. The method of claim 21, wherein a difference between the size of the first and second amplification products is indicative of germ line specific genomic instability.

23. A method for assessing infertility by detecting genomic instability comprising:
(a) obtaining a first DNA sample from at least one germ cell or testicular cell, the first DNA sample comprising at least one microsatellite locus selected from the group consisting of: Y chromosome microsatellite loci; extended mononucleotide repeat loci having at least 38 repeats; and A-rich short tandem repeats having repeating units selected from the group consisting of AAAAG, AAAAC, and AAAAT;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the amplification product being indicative of genomic instability, wherein genomic instability is indicative of infertility.

24. The method of claim 23, wherein the expected size of the amplification product is assessed by a method comprising:
(e) obtaining a second DNA sample from at least one control cell, the second DNA sample comprising the at least one microsatellite locus;
(f) contacting the second DNA sample with the first and second primers of step (b) under conditions that allow amplification of the at least one microsatellite locus to form a second amplification product;
(g) determining the size of the second amplification product, wherein the size of the second amplification product is the expected size of the amplification product of step (d).

25. The method of claim 24, wherein a difference between the size of the first and second amplification products is indicative of germ line specific genomic instability.

26. The method of claim 23, wherein the germ cell is a sperm cell.

27-33. (canceled)

34. A method for assessing risk of testicular cancer for an individual comprising:
(a) obtaining a first DNA sample from at least one germ cell of the subject, the first DNA sample comprising at least one microsatellite locus;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) obtaining a second DNA sample from at least one control cell, the second DNA sample comprising the at least one microsatellite locus;
(d) contacting the second DNA sample with the first and second primers of step (b) under conditions that allow amplification of the at least one microsatellite locus to form a second amplification product;
(e) determining the size of the first and second amplification products; and
(f) comparing the size of the second amplification product to the size of the first amplification product, a difference between the size of the first and second amplification products being indicative of germ line specific genomic instability, wherein germ line specific genomic instability is indicative of increased risk for testicular cancer.

35-39. (canceled)

40. A method for detecting genomic instability in an individual comprising:
(a) obtaining a first DNA sample from at least one testicular cell, the first DNA sample comprising at least one microsatellite locus, wherein the at least one microsatellite locus is selected from the group consisting of Y chromosome microsatellite loci, extended mononucleotide repeat loci having at least 41 repeats, MONO-27, NR-24, PENTA D, BAT-25, D7S3070, and D7S1808;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the amplification product being indicative of genomic instability.

41-50. (canceled)

51. The method of claim 40, wherein germ line specific genomic instability is indicative of infertility.

52. The method of claim 40, wherein the expected size of the amplification product is assessed by a method comprising:
(e) obtaining a second DNA sample from at least one control cell, the second DNA sample comprising the at least one microsatellite locus;
(f) contacting the second DNA sample with the first and second primers of step (b) under conditions that allow amplification of the at least one microsatellite locus to form a second amplification product;
(g) determining the size of the second amplification product, wherein the size of the second amplification product is the expected size of the amplification product of step (d).

53. A method of assessing risk of testicular cancer for an individual comprising:
(a) obtaining a first DNA sample from at least one testicular cell of the subject, the first DNA sample comprising at least one microsatellite locus, wherein the at least one microsatellite locus comprises at least one microsatellite locus selected from the group consisting of Y chromosome microsatellite loci, extended mononucleotide repeat loci having at least 41 repeats, MONO-27, NR-24, PENTA D, BAT-25, D7S3070, and D7S1808;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the first amplification product being indicative of germ line specific genomic instability, wherein germ line specific genomic instability is indicative of increased risk for testicular cancer.

54. The method of claim 53, wherein the expected size of the amplification product is assessed by a method comprising:
(e) obtaining a second DNA sample from at least one control cell, the second DNA sample comprising the at least one microsatellite locus;
(f) contacting the second DNA sample with the first and second primers of step (b) under conditions that allow amplification of the at least one microsatellite locus to form a second amplification product;
(g) determining the size of the second amplification product, wherein the size of the second amplification product is the expected size of the amplification product of step (d).

55. The method of claim 53, wherein the at least one microsatellite locus is selected from the group consisting of the extended mononucleotide repeat loci described in Table 3.

56-62. (canceled)

63. A method for detecting microsatellite instability in a putative cancer or precancerous cell, or a tumor comprising:
(a) obtaining a first DNA sample from at least one putative cancer or precancerous cell, or tumor cell, the first DNA sample comprising at least one Y chromosome microsatellite locus;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the first amplification product being indicative of microsatellite instability.

64. The method of claim 63, wherein the expected size of the amplification product is assessed by a method comprising:
(e) obtaining a second DNA sample from at least one normal cell, the second DNA sample comprising the at least one microsatellite locus;
(f) contacting the second DNA sample with the first and second primers of step (b) under conditions that allow amplification of the at least one microsatellite locus to form a second amplification product;
(g) determining the size of the second amplification product, wherein the size of the second amplification product is the expected size of the amplification product of step (d).

65. (canceled)

66. A method for monitoring genomic stability of a cultured pluripotent cell or a stem cell line comprising:
(a) obtaining a first DNA sample from at least one stem cell or at least one pluripotent cell, the first DNA sample comprising at least one microsatellite locus;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the amplification product being indicative of genomic instability.

67. The method of claim 66, wherein the expected size of the amplification product is assessed by a method comprising:
(e) obtaining a second DNA sample from at least one control cell, the second DNA sample comprising the at least one microsatellite locus;
(f) contacting the second DNA sample with the first and second primers of step (b) under conditions that allow amplification of the at least one microsatellite locus to form a second amplification product;
(g) determining the size of the second amplification product, wherein the size of the second amplification product is the expected size of the amplification product of step (d).

68-71. (canceled)

72. The method of claim 66, wherein the at least one microsatellite locus comprises at least one extended mononucleotide repeat locus.

73. The method of claim 72, wherein the at least one extended mononucleotide repeat locus is selected from the group consisting of the extended mononucleotide repeat loci described in Table 3.

74. The method of claim 72, wherein the at least one extended mononucleotide repeat locus is selected from the group consisting of the extended mononucleotide repeat loci having at least 38 repeats.

75-77. (canceled)

78. A method of monitoring exposure to mutagens or potential mutagens comprising:
(a) obtaining a first DNA sample from at least one germ cell, the first DNA sample comprising at least one microsatellite locus, wherein the at least one microsatellite locus is selected from the group consisting of Y chromosome microsatellite loci, extended mononucleotide repeat loci having at least 41 repeats, MONO-27, PENTA C, and D7S3070;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) determining the size of the first amplification product; and
(d) comparing the size of the first amplification product to the expected size of the amplification product, a difference between the size of the first amplification product and the expected size of the amplification product being indicative of genomic instability, wherein genomic instability is indicative of exposure to the mutagen or potential mutagen.

79. The method of claim 78, wherein the expected size of the amplification product is assessed by a method comprising:
(e) obtaining a second DNA sample from at least one control cell from, the second DNA sample comprising the at least one microsatellite locus;
(f) contacting the second DNA sample with the first and second primers of step (b) under conditions that allow amplification of the at least one microsatellite locus to form a second amplification product;
(g) determining the size of the second amplification product, wherein the size of the second amplification product is the expected size of the amplification product of step (d).

80. The method of claim 78, wherein the at least one microsatellite locus comprises at least one Y chromosome microsatellite locus selected from the group consisting of mononucleotide repeat loci, dinucleotide repeat loci, trinucleotide repeat loci, tetranucleotide repeat loci, and pentanucleotide repeat loci.

81-85. (canceled)

86. The method of claim 78, wherein the germ cell and control cell are obtained from an organism or cultured cells at different times.

87. The method of claim 78, wherein the germ cell is obtained from an organism or cells exposed to a mutagen and wherein the control cell is obtained from an organism or cells not exposed to the mutagen.

88. The method of claim 78, wherein the mutagen is a free radical or reactive oxygen species or substance producing a free radical or reactive oxygen species or an environmental condition that induces free radicals or a reactive oxygen species.

89. A method of monitoring exposure to mutagens or potential mutagens comprising:
(a) obtaining a first DNA sample from at least one germ cell, the first DNA sample comprising at least one microsatellite locus, wherein the at least one microsatellite locus is selected from the group consisting of Y chromosome microsatellite loci, extended mononucleotide repeat loci having at least 38 repeats, MONO-27, PENTA C, and D7S3070;
(b) contacting the first DNA sample with a first primer and a second primer that hybridize to a first DNA sequence and a second DNA sequence, respectively, wherein the first and second DNA sequences flank or partially overlap the at least one microsatellite locus, under conditions that allow amplification of the at least one microsatellite locus to form a first amplification product;
(c) obtaining a second DNA sample from at least one control cell prior to obtaining the first DNA sample of step (a), the second DNA sample comprising the at least one microsatellite locus;
(d) contacting the second DNA sample with the first and second primers of step (b) under conditions that allow amplification of the at least one microsatellite locus to form a second amplification product;
(e) determining the size of the first and second amplification products; and
(f) comparing the size of the first amplification product to the size of the second amplification product, a difference between the size of the first amplification product and the size of the second amplification product being indicative of genomic instability, wherein genomic instability is indicative of exposure to the mutagen or potential mutagen.

90-95. (canceled)

96. The method of claim 89, wherein the germ cell is obtained from an organism or cells exposed to a mutagen and wherein the control cell is obtained from an organism or cells not exposed to the mutagen.

97. The method of claim 89, wherein the mutagen is a free radical or reactive oxygen species or substance producing a free radical or reactive oxygen species or an environmental condition that induces free radicals or a reactive oxygen species.

98-102. (canceled)


SEQUENCE LISTING (166 sequences)

SEQ ID NO: 1 - DNA, 26 bp, Homo sapiens
gtggtcttctacttgtgtcaatacag

SEQ ID NO: 2 - DNA, 23 bp, Homo sapiens
gaactcaagtccaaaaaatgagg

SEQ ID NO: 3 - DNA, 26 bp, Homo sapiens
atttatattttacacatttttgggcc

SEQ ID NO: 4 - DNA, 23 bp, Homo sapiens
tgacagtaaaatgaaaacattgc

SEQ ID NO: 5 - DNA, 22 bp, Homo sapiens
attagcatgggtgacagagcta

SEQ ID NO: 6 - DNA, 22 bp, Homo sapiens
ccaattacatagtcctcctttc

SEQ ID NO: 7 - DNA, 24 bp, Homo sapiens
ttcaatcatacacccatatctgtc

SEQ ID NO: 8 - DNA, 23 bp, Homo sapiens
attatagagggataggtaggcag

SEQ ID NO: 9 - DNA, 25 bp, Homo sapiens
ccaactctcatctgtattatctatg

SEQ ID NO: 10 - DNA, 27 bp, Homo sapiens
attttatccctgagtagcagaagaatg

SEQ ID NO: 11 - DNA, 21 bp, Homo sapiens
tcgagttgttatggttttagg

SEQ ID NO: 12 - DNA, 24 bp, Homo sapiens
atttggcttggaattcttttaccc

SEQ ID NO: 13 - DNA, 20 bp, Homo sapiens
tggggaatagttgaacggta

SEQ ID NO: 14 - DNA, 25 bp, Homo sapiens
attgcaacaagagtgaaactccatt

SEQ ID NO: 15 - DNA, 23 bp, Homo sapiens
attgactatgggcgtgagtgcat

SEQ ID NO: 16 - DNA, 22 bp, Homo sapiens
agaccctgtcattcacagatga

SEQ ID NO: 17 - DNA, 28 bp, Homo sapiens
actactgagtttctgttatagtgttttt

SEQ ID NO: 18 - DNA, 22 bp, Homo sapiens
gtcaatctctgcacctggaaat

SEQ ID NO: 19 - DNA, 23 bp, Homo sapiens
atttagaggcagtcatcgcagtg

SEQ ID NO: 20 - DNA, 22 bp, Homo sapiens
acctaccaatcccattccttag

SEQ ID NO: 21 - DNA, 24 bp, Homo sapiens
cggagtcgctggcacagttctatt

SEQ ID NO: 22 - DNA, 25 bp, Homo sapiens
tcgcgtttacaaacaagaaaagtgt

SEQ ID NO: 23 - DNA, 24 bp, Homo sapiens
tgactacttttgacttcagccagt

SEQ ID NO: 24 - DNA, 24 bp, Homo sapiens
aaccattcaacatttttaaccctt

SEQ ID NO: 25 - DNA, 20 bp, Homo sapiens
tcgcctccaagaatgtaagt

SEQ ID NO: 26 - DNA, 24 bp, Homo sapiens
atttctgcattttaactatggctc

SEQ ID NO: 27 - DNA, 20 bp, Homo sapiens
ccattgctgaattttacctc

SEQ ID NO: 28 - DNA, 20 bp, Homo sapiens
attgtgccattgcattccaa

SEQ ID NO: 29 - DNA, 24 bp, Homo sapiens
tgtgaaccacctatgaattgcaga

SEQ ID NO: 30 - DNA, 26 bp, Homo sapiens
attgcttgcagtgagcagagatcgtt

SEQ ID NO: 31 - DNA, 24 bp, Homo sapiens
catggcattggggacatgaacaca

SEQ ID NO: 32 - DNA, 24 bp, Homo sapiens
cactgagcgcttctagggacttct

SEQ ID NO: 33 - DNA, 24 bp, Homo sapiens
cagcctaggtgacagagcaagaca

SEQ ID NO: 34 - DNA, 24 bp, Homo sapiens
atttgcctaacctatggtcataac

SEQ ID NO: 35 - DNA, 28 bp, Homo sapiens
gaggctgaggcaggagaatggcgtgaac

SEQ ID NO: 36 - DNA, 28 bp, Homo sapiens
cgctgacgcagaacctgaaattgtgatt

SEQ ID NO: 37 - DNA, 26 bp, Homo sapiens
tatcctagcttggcctgtttaagacc

SEQ ID NO: 38 - DNA, 22 bp, Homo sapiens
tgaggcaggagaatggcgtgaa

SEQ ID NO: 39 - DNA, 28 bp, Homo sapiens
tctcatttgagtggtggaagtgactggt

SEQ ID NO: 40 - DNA, 23 bp, Homo sapiens
tattctttcgggatgtaatctct

SEQ ID NO: 41 - DNA, 28 bp, Homo sapiens
aggctgaagcaggagaatcacttaaaac

SEQ ID NO: 42 - DNA, 25 bp, Homo sapiens
gccaagtgtcgcttgtaattctatt

SEQ ID NO: 43 - DNA, 28 bp, Homo sapiens
ctaacttcccagcaacttcctttacact

SEQ ID NO: 44 - DNA, 24 bp, Homo sapiens
attgggcagacactgaactagctt

SEQ ID NO: 45 - DNA, 28 bp, Homo sapiens
cagcctaggtaacagagcaagacctttg

SEQ ID NO: 46 - DNA, 23 bp, Homo sapiens
gtttgcgtgatttgcgtggactt

SEQ ID NO: 47 - DNA, 26 bp, Homo sapiens
tcagcagctgaaagaaatctgagtac

SEQ ID NO: 48 - DNA, 22 bp, Homo sapiens
gcgatacccaaagtcaatagtc

SEQ ID NO: 49 - DNA, 24 bp, Homo sapiens
gaagctgcagtaagccgagattgt

SEQ ID NO: 50 - DNA, 24 bp, Homo sapiens
gccctcttaactcccatgacattc

SEQ ID NO: 51 - DNA, 20 bp, Homo sapiens
catttcttctgcccccatga

SEQ ID NO: 52 - DNA, 26 bp, Homo sapiens
atttgacagctgaaaaggtgcagatg

SEQ ID NO: 53 - DNA, 21 bp, Homo sapiens
gaggagacagccagggatata

SEQ ID NO: 54 - DNA, 27 bp, Homo sapiens
atttctctataacctctctccctatct

SEQ ID NO: 55 - DNA, 24 bp, Homo sapiens
ggaggaaaagtcttaaacgtgaat

SEQ ID NO: 56 - DNA, 24 bp, Homo sapiens
attggccttgatgtgtttgttact

SEQ ID NO: 57 - DNA, 20 bp, Homo sapiens
gccgatcctgaagcaatagc

SEQ ID NO: 58 - DNA, 23 bp, Homo sapiens
attccccttggtggtgtcatcct

SEQ ID NO: 59 - DNA, 20 bp, Homo sapiens
gtttgcatgtgaacaggtca

SEQ ID NO: 60 - DNA, 23 bp, Homo sapiens
attggcaggcaggtagatagaca

SEQ ID NO: 61 - DNA, 20 bp, Homo sapiens
ggctgcagggcataacatta

SEQ ID NO: 62 - DNA, 24 bp, Homo sapiens
attctatgactttgcgcttcagga

SEQ ID NO: 63 - DNA, 20 bp, Homo sapiens
gcacagaacaggcacttagg

SEQ ID NO: 64 - DNA, 18 bp, Homo sapiens
cgctcaaacgtgaggttg

SEQ ID NO: 65 - DNA, 32 bp, Homo sapiens
attgcaacttatatgtatttttgtatttcatg

SEQ ID NO: 66 - DNA, 28 bp, Homo sapiens
accaaattgtgttcatgagtatagtttc

SEQ ID NO: 67 - DNA, 33 bp, Homo sapiens
gccctagtggatgataagaataatcagtatgtg

SEQ ID NO: 68 - DNA, 30 bp, Homo sapiens
ggacagatgataaatacataggatggatgg

SEQ ID NO: 69 - DNA, 19 bp, Homo sapiens
ccctgggctctgtaaagaa

SEQ ID NO: 70 - DNA, 24 bp, Homo sapiens
atcagagcttaaactgggaagctg

SEQ ID NO: 71 - DNA, 26 bp, Homo sapiens
attaccaacatgaaagggtaccaata

SEQ ID NO: 72 - DNA, 33 bp, Homo sapiens
tgggttattaattgagaaaactccttacaattt

SEQ ID NO: 73 - DNA, 20 bp, Homo sapiens
ttcttgagcccagaaggtta

SEQ ID NO: 74 - DNA, 27 bp, Homo sapiens
attctaccagcaacaacacaaataaac

SEQ ID NO: 75 - DNA, 22 bp, Homo sapiens
atatgtgagtcaattccccaag

SEQ ID NO: 76 - DNA, 26 bp, Homo sapiens
tgtattagtcaatgttctccagagac

SEQ ID NO: 77 - DNA, 21 bp, Homo sapiens
gtgattcccattggcctgttc

SEQ ID NO: 78 - DNA, 22 bp, Homo sapiens
attcctgtgggctgaaaagctc

SEQ ID NO: 79 - DNA, 19 bp, Homo sapiens
actgcagtccaatctgggt

SEQ ID NO: 80 - DNA, 21 bp, Homo sapiens
atgaaatcaacagaggcttgc

SEQ ID NO: 81 - DNA, 19 bp, Homo sapiens
gaaggtcgaagctgaagtg

SEQ ID NO: 82 - DNA, 27 bp, Homo sapiens
attagaattctttaatctggacacaag

SEQ ID NO: 83 - DNA, 24 bp, Homo sapiens
ccggaggtaaaggtgtcttaaagt

SEQ ID NO: 84 - DNA, 22 bp, Homo sapiens
atttcctgtgtcagaccctgtt

SEQ ID NO: 85 - DNA, 24 bp, Homo sapiens
gggggtctaagagcttgtaaaaag

SEQ ID NO: 86 - DNA, 29 bp, Homo sapiens
gtttgtgtgtgcatctgtaagcatgtatc

SEQ ID NO: 87 - DNA, 21 bp, Homo sapiens
atgttggtcaggctgactatg

SEQ ID NO: 88 - DNA, 24 bp, Homo sapiens
gattccacatttatcctcattgac

SEQ ID NO: 89 - DNA, 26 bp, Homo sapiens
attacagaagtctgggatgtggagga

SEQ ID NO: 90 - DNA, 19 bp, Homo sapiens
ggcagcccaaaaagacaga

SEQ ID NO: 91 - DNA, 23 bp, Homo sapiens
ggtgattttcctctttggtatcc

SEQ ID NO: 92 - DNA, 26 bp, Homo sapiens
agccacagtttacaacatttgtatct

SEQ ID NO: 93 - DNA, 24 bp, Homo sapiens
tataattaggtcccagatcactta

SEQ ID NO: 94 - DNA, 24 bp, Homo sapiens
ggcaatgtttaaagacatggatac

SEQ ID NO: 95 - DNA, 24 bp, Homo sapiens
aaacacagtgagactccctatcta

SEQ ID NO: 96 - DNA, 24 bp, Homo sapiens
acaggacagagatggcacggacag

SEQ ID NO: 97 - DNA, 24 bp, Homo sapiens
ctgctgttgcatcgcggcccaatg

SEQ ID NO: 98 - DNA, 24 bp, Homo sapiens
aagaagcccctctcctccggtctc

SEQ ID NO: 99 - DNA, 24 bp, Homo sapiens
aggcatgggcaaggacttgatgtc

SEQ ID NO: 100 - DNA, 24 bp, Homo sapiens
ctggatgttagccgtttgtcagag

SEQ ID NO: 101 - DNA, 24 bp, Homo sapiens
ggtttgcttgaggccagaacttca

SEQ ID NO: 102 - DNA, 24 bp, Homo sapiens
ctcatagcagccttaaattactga

SEQ ID NO: 103 - DNA, 24 bp, Homo sapiens
agcctgggcgacagagcaagactc

SEQ ID NO: 104 - DNA, 24 bp, Homo sapiens
caagggcagcatcattatgacaac

SEQ ID NO: 105 - DNA, 28 bp, Homo sapiens
tgtgtgcaaattgtgagggaggtaggta

SEQ ID NO: 106 - DNA, 24 bp, Homo sapiens
agcggggtgcggtggctcatatct

SEQ ID NO: 107 - DNA, 24 bp, Homo sapiens
ctgaggcaggagaatggagagtag

SEQ ID NO: 108 - DNA, 24 bp, Homo sapiens
ctctgctacccgggttcaaacagt

SEQ ID NO: 109 - DNA, 28 bp, Homo sapiens
gaggctgaggcaggagaatggcgtgaac

SEQ ID NO: 110 - DNA, 28 bp, Homo sapiens
cgctgacgcagaacctgaaattgtgatt

SEQ ID NO: 111 - DNA, 24 bp, Homo sapiens
aggttgcagtgagccaggatcata

SEQ ID NO: 112 - DNA, 24 bp, Homo sapiens
atcacatcatctgtcccacctaac

SEQ ID NO: 113 - DNA, 24 bp, Homo sapiens
tgggcgacagagcgagactccgtc

SEQ ID NO: 114 - DNA, 24 bp, Homo sapiens
cagcggcccataaattctatgtta

SEQ ID NO: 115 - DNA, 28 bp, Homo sapiens
ctaacttcccagcaacttcctttacact

SEQ ID NO: 116 - DNA, 24 bp, Homo sapiens
attgggcagacactgaactagctt

SEQ ID NO: 117 - DNA, 28 bp, Homo sapiens
gggagaaccttgctgtctttcagataat

SEQ ID NO: 118 - DNA, 24 bp, Homo sapiens
agggctcctggaatatggttgtac

SEQ ID NO: 119 - DNA, 28 bp, Homo sapiens
aacctccaccttcccagctcaagtgaca

SEQ ID NO: 120 - DNA, 23 bp, Homo sapiens
ggcgacagcgagactccgtctca

SEQ ID NO: 121 - DNA, 24 bp, Homo sapiens
ctgaggcaggagaatggcgtgaac

SEQ ID NO: 122 - DNA, 28 bp, Homo sapiens
atgatgctggcctcataaaaagagttag

SEQ ID NO: 123 - DNA, 26 bp, Homo sapiens
tatcctagcttggcctgtttaagacc

SEQ ID NO: 124 - DNA, 22 bp, Homo sapiens
tgaggcaggagaatggcgtgaa

SEQ ID NO: 125 - DNA, 26 bp, Homo sapiens
tttaatatacctgctgatcaatgata

SEQ ID NO: 126 - DNA, 21 bp, Homo sapiens
gacacatgggatcatagcaaa

SEQ ID NO: 127 - DNA, 24 bp, Homo sapiens
ttgggcgacagagcaagacgactc

SEQ ID NO: 128 - DNA, 26 bp, Homo sapiens
atttggtcagtgggggctctgttaag

SEQ ID NO: 129 - DNA, 26 bp, Homo sapiens
tcagcagctgaaagaaatctgagtac

SEQ ID NO: 130 - DNA, 22 bp, Homo sapiens
gcgatacccaaagtcaatagtc

SEQ ID NO: 131 - DNA, 24 bp, Homo sapiens
gaagctgcagtaagccgagattgt

SEQ ID NO: 132 - DNA, 24 bp, Homo sapiens
gccctcttaactcccatgacattc

SEQ ID NO: 133 - DNA, 22 bp, Homo sapiens
agcctgggcgacagagcgagtc

SEQ ID NO: 134 - DNA, 23 bp, Homo sapiens
ctcggggctcgggagatgagtga

SEQ ID NO: 135 - DNA, 28 bp, Homo sapiens
cagcctaggtaacagagcaagacctttg

SEQ ID NO: 136 - DNA, 23 bp, Homo sapiens
gtttgcgtgatttgcgtggactt

SEQ ID NO: 137 - DNA, 23 bp, Homo sapiens
ctcctgcctcatcctcccgagta

SEQ ID NO: 138 - DNA, 24 bp, Homo sapiens
ccgagatcacgccactgcactcta

SEQ ID NO: 139 - DNA, 28 bp, Homo sapiens
tctcatttgagtggtggaagtgactggt

SEQ ID NO: 140 - DNA, 23 bp, Homo sapiens
tattctttcgggatgtaatctct

SEQ ID NO: 141 - DNA, 26 bp, Homo sapiens
cccgtctctactaaaaatactaaaac

SEQ ID NO: 142 - DNA, 28 bp, Homo sapiens
aaaccaacaataaggcaacctcttagtc

SEQ ID NO: 143 - DNA, 28 bp, Homo sapiens
tgccagagtagggtggtccatggtactt

SEQ ID NO: 144 - DNA, 25 bp, Homo sapiens
gcccaaaatgtgtttagttagcttc

SEQ ID NO: 145 - DNA, 28 bp, Homo sapiens
aggctgaagcaggagaatcacttaaaac

SEQ ID NO: 146 - DNA, 25 bp, Homo sapiens
gccaagtgtcgcttgtaattctatt

SEQ ID NO: 147 - DNA, 26 bp, Homo sapiens
gaatcttgtttcggcctttgacctta

SEQ ID NO: 148 - DNA, 25 bp, Homo sapiens
cgagatcacgccaccgcactctagc

SEQ ID NO: 149 - DNA, 27 bp, Homo sapiens
aaatctacccagctctgtaacgagaga

SEQ ID NO: 150 - DNA, 28 bp, Homo sapiens
aagctctgtttggcaagtgttaattgta

SEQ ID NO: 151 - DNA, 28 bp, Homo sapiens
ttggaatgtattctctgggtttggcagt

SEQ ID NO: 152 - DNA, 27 bp, Homo sapiens
ttcaggaggctgaggtgggaggattgt

SEQ ID NO: 153 - DNA, 22 bp, Homo sapiens
acctaggcaataccatctaaga

SEQ ID NO: 154 - DNA, 24 bp, Homo sapiens
gttgcctgttcactctgatagtct

SEQ ID NO: 155 - DNA, 23 bp, Homo sapiens
agcctgggtgacagagcgagact

SEQ ID NO: 156 - DNA, 28 bp, Homo sapiens
ttagagttatttgttgggatgagaatct

SEQ ID NO: 157 - DNA, 22 bp, Homo sapiens
ctgggcgacagagcgagactcc

SEQ ID NO: 158 - DNA, 26 bp, Homo sapiens
tctcctgccttagcctcccgagtagc

SEQ ID NO: 159 - DNA, 27 bp, Homo sapiens
tcctctccctaaaaagctccccctaag

SEQ ID NO: 160 - DNA, 28 bp, Homo sapiens
aggtcaaggctgcggtaagctgtgatcg

SEQ ID NO: 161 - DNA, 28 bp, Homo sapiens
tccccactttgtcctgcacactcctacc

SEQ ID NO: 162 - DNA, 23 bp, Homo sapiens
gggcgacagagcgagactccgtc

SEQ ID NO: 163 - DNA, 28 bp, Homo sapiens
aagatttaatagacatgcgcagaacact

SEQ ID NO: 164 - DNA, 23 bp, Homo sapiens
ccagcctgggcaaaagagcaagt

SEQ ID NO: 165 - DNA, 28 bp, Homo sapiens
acaaacatgaaaaggcaaatgatagaac

SEQ ID NO: 166 - DNA, 27 bp, Homo sapiens
agaggttgcagtgagccaagattgtag